(19) United States
(12) Patent Application Publication
(10) Pub. No.: US 2005/0049206 A1
(43) Pub. Date: Mar. 3, 2005

(54) Compositions of flavonoids and flavonoid-containing extracts and the treatment of diseases

(76) Inventors: Bang Qiang Gong, Shanghai (CN); Ren Jin, Shanghai (CN); Han Xing Chun, Shanghai (CN)

Correspondence Address: FENWICK & WEST LLP, SILICON VALLEY CENTER, 801 CALIFORNIA STREET, MOUNTAIN VIEW, CA, 94041, US

(21) Appl. No.: 10/746,632

(22) Filed: Dec. 23, 2003

(30) Foreign Application Priority Data
Sep. 1, 2003 (CN) 03150702.6

(51) Int. Cl. A61K 31/7048; A61K 31/353

(52) U.S. Cl. 514/27; 514/456

(57) ABSTRACT

In this invention, we describe a group of flavonoids and flavonoid-containing extracts that have pharmaceutical properties which are useful in the medicinal therapy of fibrotic diseases for the treatment or reparation and prevention of fibrotic lesional tissues. Representative flavonoids and flavonoid-containing extracts have the active compositions of the below formula. 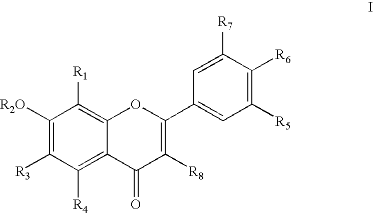 Those compositions can be extracted and purified from the botanicals, including Scutellaria baicalensis Georgi, Scutellaria scordifolia Fisch,Oroxylum indicum(L.) Vent, Plantago major L. The compositions of the invention are novel as an anti-fibrotic drugs, as agents for treating fibrosis.

### 1. CROSS REFERENCE TO RELATED APPLICATION

This application claims the benefit under 35 U.S.C. section 119 of Chinese patent application serial number 03150702.6, filed Sep. 1, 2003, the entire contents of which are incorporated by reference for all purposes.

### 2. FIELD OF THE INVENTION

The present invention relates to the field of medicine, and more specifically to compositions and methods useful for the treatment of fibrotic lesional tissues and the prevention of fibrotic lesions. The compositions comprise one or more flavonoids and flavonoid-containing extracts.

### 3. BACKGROUND OF THE INVENTION

At present, no effective pharmacological agent or composition has been available on the market for the prevention or removal of pathologic fibrotic lesions of the lungs, or resulting from myocardial infarction, myocardial degeneration, arteriosclerosis, or musculoskeletal diseases, or found within prostate glands, and other lesional fibroses.

For example, powerful anti-inflammatory glucocorticoids (hormones relating to carbohydrate metabolism) such as hydrocortisone or prednisolone administered in very large doses have repeatedly been shown to be ineffective against fibrotic disease. These glucocorticoids could not arrest or remove such life-threatening fibrotic lesions. The glucocorticoids may be effective, however, as anti-inflammatory agents under such condition that they may temporarily ameliorate the secondary acute inflammation flare-ups, which intermittently occur in tissues or organs damaged by fibrotic disease. Indeed, excessive and prolonged administration of glucocorticoids in pulmonary fibrotic disease may cause destruction of tissues, due to fibrosis or an exacerbation and acceleration of the fibrotic destruction.

Antopol (1950) was the first investigator who found that the anti-inflammatory glucocorticoids readily enhance fibrotic degeneration of lung tissues. Similarly, the non-steroidal anti-inflammatory agents such as aspirin, salicylates, phenylbutazone, indomethacin, various phenylacetic acid derivatives, and the like have also failed to arrest formation of, or cause repair of progressive, chronic fibrotic damage to lung tissues, prostatic tissues, musculoskeletal tissues, etc. Accordingly, it is a principal object of the present invention to provide compositions for the reparation and prevention of fibrotic lesional tissue.

It is an additional object of the invention to provide such compositions that comprise one or more from the group of flavonoids and flavonoid-containing extracts we described here.

Other objects of the present invention, as well as particular features and advantages thereof, will be elucidated in, or be apparent from the following description.

### 4. SUMMARY OF THE INVENTION

The present invention overcomes the limitations of the prior technology by providing, in a preferred embodiment, drugs having pharmacological properties that are useful in the medicinal therapy of fibrotic disease for the treatment or reparation and prevention of fibrotic lesional tissues, such drugs including as the active ingredient one or more from the group of flavonoids and flavonoid-containing extracts we described herein. The compositions of this invention are novel as an anti-fibrotic drug, namely, as an agent for treating and preventing fibrosis. Any existing compounds have not been shown to be effective for the reparation and prevention of fibrotic lesions on the market. The active ingredient exerts an anti-fibrotic activity quite dissimilar to and independent of fibrinolytic activity.

In one aspect, the invention provides compositions and methods for the reparation of or prophylaxis against fibrotic lesional tissue. The methods of the invention comprise administering to a mammal a pharmaceutical composition comprising one or more compounds of formula I 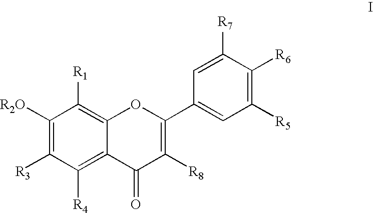
formula I
or a prodrug thereof or a pharmaceutically acceptable salts thereof, in admixture with a pharmaceutically acceptable carrier wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen, hydroxy, methyl or a straight or branched C₁-C₅ alkoxy;
R₆ is hydrogen, hydroxy, methyl or a straight or branched C₁-C₅ alkoxy;
R₇ is hydrogen, hydroxy, methyl or a straight or branched C₁-C₅ alkoxy;
R₈ is hydrogen, hydroxy or a straight or branched C₁-C₅ alkoxy; and
R₂ is hydrogen, a straight or branched C₁-C₅ alkyl, a protected or unprotected monosaccharide (pyranose or furanose), disaccharide, trisaccharide and their analogues or derivates (sugar alcohol, sugar acid) such as glucose, glucuronic acid, galactose, mannose, allose, idose, allulose, altrose, gulose, tagatose, talose, ribose, arabinose, xylose, lyxose, sorbose, amylomaltose, cellulose, lactose, sucrose.

In another aspect, the invention provides pharmaceutical compositions. The pharmaceutical compositions comprise one or more compounds of formula I 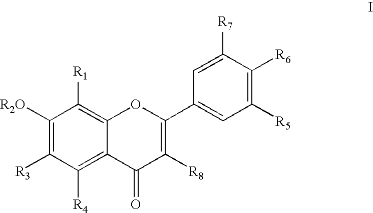
formula I
or a prodrug thereof or a pharmaceutically acceptable salt, hydrate, or solvate thereof, in admixture with a pharmaceutically acceptable carrier wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen, hydroxy, methyl or a straight or branched C₁-C₅ alkoxy;
R₆ is hydrogen, hydroxy, methyl or a straight or branched C₁-C₅ alkoxy;
R₇ is hydrogen, hydroxy, methyl or a straight or branched C₁-C₅ alkoxy;
R₈ is hydrogen, hydroxy or a straight or branched C₁-C₅ alkoxy; and
R₂ is hydrogen, a straight or branched C₁-C₅ alkyl, a protected or unprotected monosaccharide (pyranose or furanose), disaccharide, trisaccharide and their analogues or derivates (sugar alcohol, sugar acid) such as glucose, glucuronic acid, galactose, mannose, allose, idose, allulose, altrose, gulose, tagatose, talose, ribose, arabinose, xylose, lyxose, sorbose, amylomaltose, cellulose, lactose, sucrose.

In another aspect, the invention provides a method for the reparation of or prophylaxis against fibrotic lesional tissue the method comprising administering to a mammal extracts or fractions of extracts from botanicals selected from the group consisting of Scutellaria baicalensis Georgi, Scutellaria scordifolia Fisch, Oroxylum indicum(L.) Vent, and Plantago major L. pharmaceutical.

### 5. BRIEF DESCRIPTION OF THE FIGURES

FIG. 1 shows the normal lung tissue of Sprague-Dawley (S-D) rat.

FIG. 2 shows the lung tissue 28 days after intratracheal instillation of Bleomycin (BL) in S-D rats. The S-D rats in the BL group showed multifocal lesions or fibrosis containing an accumulation of extracellar fibers and a cellular infiltrate of predominantly mononuclear cells. These lesions were mainly located in peribronchiolar, perivascular and the alveolar interstitium, and some lungs showed considerable mononuclear cell alveolitis.

FIG. 3 shows the lung tissue 28 days after treatment with baicalin. Lungs from the baicalin-treated rats showed lesions in the same locations but of a more mild nature than of the BL group. The lesions in the lungs from the baicalin-treated group had less extracellular fibers, but some lobes showed a moderate degree of mononuclear cell alveolitis.

FIG. 4 shows the lung tissue 28 days after treatment with baicalin. Lungs from the baicalin-treated rats showed lesions in the same locations but of a more mild nature than of the BL group. The lesions in the lungs from the baicalin-treated group had less extracellular fibers, but some lobes showed a moderate degree of mononuclear cell alveolitis.

FIG. 5 illustrates representative parenchymal photomicrographs (×10) from normal S-D rat lung tissue at day 28. Note the existing normal lung alveoli in lung tissue. The tissue stained with hematoxylin and eosin (HE).

FIG. 6 shows representative parenchymal photomicrographs (×10) from the lungs of 28 days after intratracheal instillation of Bleomycin (BL) in S-D rats. The S-D rats in the BL group showed the severe diffuse lesions and or fibrosis containing an accumulation of extracellar fibers and a cellular infiltrate of predominantly mononuclear cells. These lesions were mainly located in peribronchiolar, perivascular and the alveolar interstitium, and some lungs showed considerable mononuclear cell alveolitis. The tissue was stained with HE.

FIG. 7 shows representative parenchymal photomicrographs (×10) from the lungs of baicalin-treated BL-induced rats. They showed lesions in the same locations but of a more mild nature than those of the BL group. The lesions in the lungs from the baicalin-treated group had less extracellular fibers, the thin interalveolar septa in proximal acinus and lack of inflammatory cells but some lobes showed a moderate degree of mononuclear cell alveolitis.

FIG. 8 illustrates a representative macroscopic view of cirrhotic rat livers treated (right panel, n=10) and non-treated (left panel, n=10) with 450 mg/kg baicalin showing remarkable differences in the entire granular texture of the liver, characteristic of micronodular cirrhosis (P<0.001).

FIG. 9 illustrates the relative collagen content (per g of liver), as measured by hydroxyproline biochemical determinations; P<0.05. The hydroxyproline level from the liver of baicalin-treated group was significantly lower than the non-treated one.

FIG. 10 illustrates functional hepatic tests for each rat in the study, which showed a strong improvement. Notably, AST decreased over 3-fold in baicalin-treated animals (P<0.001). ALT decreased over 2-fold (P<0.001).

### 6. DETAILED DESCRIPTION OF THE INVENTION

I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3^{rd} Ed.” Vols. A and B, Plenum Press, New York. The practice of the present invention will employ, unless otherwise indicated, conventional methods of mass spectroscopy, protein chemistry, biochemistry, recombinant DNA techniques and pharmacology, within the skill of the art.

Where chiral centers occur in the compounds having the R₁, R₂, R₃ and R₄ moieties as defined above, the invention includes the enantiomeric compounds resulting from the chiral center as well as racemic mixtures thereof.

As used herein, the term “halogen” includes fluorine, chlorine, bromine, and iodine.

As used herein, “lower alkyl” means a straight chain or branched, saturated or unsaturated chain having from 1 to 10 carbon atoms. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, and n-hexyl, and longer alkyl groups, such as heptyl, and octyl. An alkyl group can be unsubstituted or substituted. Unsaturated alkyl groups include alkenyl groups and alkynyl groups, discussed below.

As used herein, an “alkenyl group” includes a monovalent unbranched or branched hydrocarbon chain having one or more double bonds therein. The double bond of an alkenyl group can be unconjugated or conjugated to another unsaturated group. Suitable alkenyl groups include, but are not limited to, (C₂-C₈)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted.

As used herein, “alkynyl group” includes a monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted.

The terms “trifluoromethyl,” “sulfonyl,” and “carboxyl” include CF₃, SO₃H, and CO₂H, respectively.

The term “alkoxy” as used herein includes —O-(alkyl), wherein alkyl is defined above.

As used herein, “alkoxyalkoxy” includes —O-(alkyl)-O-(alkyl), wherein each “alkyl” is independently an alkyl group defined above.

As used herein, “alkoxycarbonyl” includes —C(O)O-(alkyl), wherein alkyl is defined above.

As used herein, “alkoxycarbonylalkyl” includes -(alkyl)-C(O)O-(alkyl), wherein alkyl is defined above.

As used herein, “alkoxyalkyl” means -(alkyl)-O-(alkyl), wherein each “alkyl” is independently an alkyl group defined above.

As used herein, “aryl” includes a carbocyclic or heterocyclic aromatic group containing from 5 to 30 ring atoms. The ring atoms of a carbocyclic aromatic group are all carbon atoms, and include, but are not limited to, phenyl, tolyl, anthracenyl, fluorenyl, indenyl, azulenyl, and naphthyl, as well as benzo-fused carbocyclic moieties such as 5,6,7,8-tefrahydronaphthyl. A carbocyclic aromatic group can be unsubstituted or substituted. Preferably, the carbocyclic aromatic group is a phenyl group. The ring atoms of a heterocyclic aromatic group contains at least one heteroatom, preferably 1 to 3 heteroatoms, independently selected from nitrogen, oxygen, and sulfur. Illustrative examples of heterocyclic aromatic groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phenyl, isoxazolyl, indolyl, oxetanyl, azepinyl, piperazinyl, morpholinyl, dioxanyl, thietanyl and oxazolyl. A heterocyclic aromatic group can be unsubstituted or substituted. Preferably, a heterocyclic aromatic is a monocyclic ring, wherein the ring comprises 2 to 5 carbon atoms and 1 to 3 heteroatoms.

As used herein, “aryloxy” includes —O-aryl group, wherein aryl is as defined above. An aryloxy group can be unsubstituted or substituted.

As used herein, “arylalkyl” includes -(alkyl)-(aryl), wherein alkyl and aryl are defined above.

As used herein, “arylalkyloxy” includes —O-(alkyl)-(aryl), wherein alkyl and aryl are defined above.

As used herein, “cycloalkyl” includes a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted. Preferably, the cycloalkyl group is a monocyclic ring or bicyclic ring.

As used herein, “cycloalkyloxy” includes —O-(cycloalkyl), wherein cycloalkyl is defined above.

As used herein, “cycloalkylalkyloxy” includes —O-(alkyl)-(cycloalkyl), wherein cycloalkyl and alkyl are defined above.

Herein, the term “anti-fibro”, “anti-fibrotic” or “anti-fibrosis” refers to the medicinal treatment or reparations and/or prevention of pathological polymerization of collagen in lung fibrosis, keloid, myocarditis, arteriosclerosis, prostatic hypertrophy, collagen disease, scar, wrinkle, etc., and reparation as well normalization of the existing pathological fibrotic tissues.

6.1. The Compounds that have Therapeutic Usage in the Invention

Preferred compounds which are utilized for the treatment of pulmonary fibrosis in the present invention include a group of flavones and their derivatives as the below formula 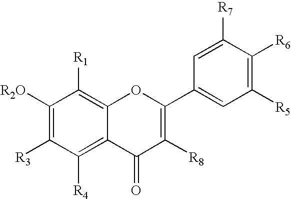
wherein
R₁ is hydrogen, hydroxy or a straight or branched C₁-C₅ alkoxy.
R₃ is hydrogen, hydroxy or a straight or branched C₁-C₅ alkoxy.
R₄ is hydrogen, hydroxy or a straight or branched C₁-C₅ alkoxy.
R₅ is hydrogen, hydroxy, methyl or a straight or branched C₁-C₅ alkoxy.
R₆ is hydrogen, hydroxy, methyl or a straight or branched C₁-C₅ alkoxy.
R₇ is hydrogen, hydroxy, methyl or a straight or branched C₁-C₅ alkoxy.
R₈ is hydrogen, hydroxy or a straight or branched C₁-C₅ alkoxy.
R₂ is hydrogen, a straight or branched C₁-C₅ alkyl, a protected or unprotected monosaccharide (pyranose or furanose), disaccharide, trisaccharide and their analogues or derivates (sugar alcohol, sugar acid) such as glucose, glucuronic acid, galactose, mannose, allose, idose, allulose, altrose, gulose, tagatose, talose, ribose, arabinose, xylose, lyxose, sorbose, amylomaltose, cellulose, lactose, sucrose.

Wherein R₂ is, for example, as in Table 1:

TABLE 1
H
Me
Bn

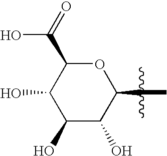

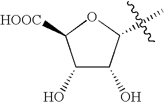

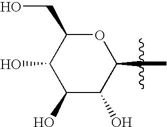

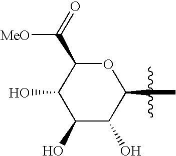

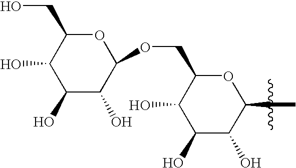

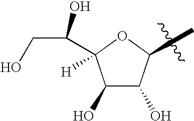

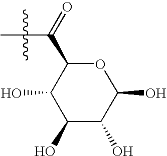

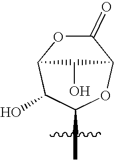

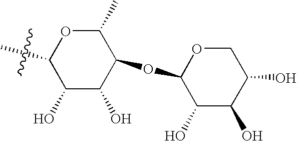

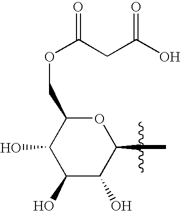

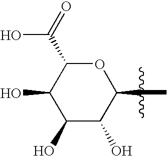

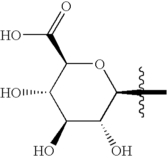

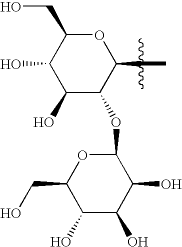

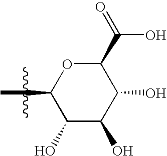

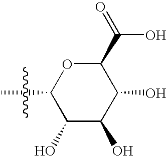

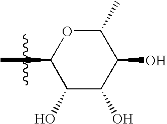

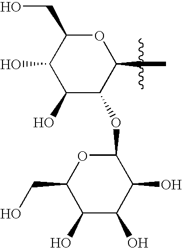

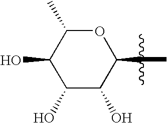

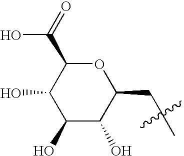

The typical specific flavones which can be utilized in the method of the invention include, for example those listed in Table 2.

TABLE 2

| Structure | Reference |
|---|---|
| 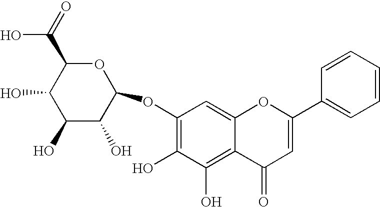 | J.Chin.Chem.Soc.(Taipei); 47; 1; 2000; 247-252. Baicalin can be isolated from Scutellaria baicalensis Georgi, Scutellaria scordifolia Fisch, Oroxylum indicum(L.) Vent., Plantago major L. etc. |
| Baicalin | |
| (2S,3S,4S,5R,6S)-6-(5,6-dihydroxy-4-oxo-2-phenyl-4H-chromen- | |
| 7-yloxy)-tetrahydro-3,4,5-trihydroxy-2H-pyran-2-carboxylic acid | |
| 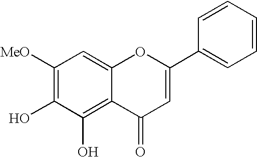 | Indian J.Chem.Sect.B; 36; 1; 1997; 104-106. |
| 5,6-Dihydroxy-7-methoxy-2-phenyl-4H-chromen-4-one | |
| 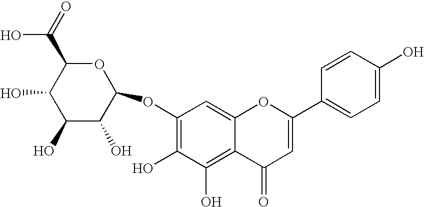 | Helv.Chim.Acta; 81; 11; 1998; 2062-2071. |
| (2S,3S,4S,5R,6S)-6-(5,6-dihydroxy-2-(4-hydroxyphenyl)-4- | |
| oxo-4H-chromen-7-yloxy)-tetrahydro-3,4,5-trihydroxy-2H- | |
| pyran-2-carboxylic acid | |
| 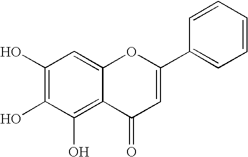 | J.Nat.Prod.; 61; 11; 1998; 1413-1415. |
| Baicalein | |
| 5,6,7-Trihydroxy-2-phenyl-4H-chromen-4-one | |
| 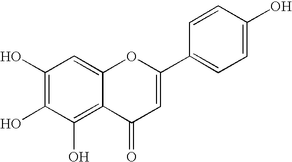 | J.Chem.Soc.; 1965; 5651-5657. |
| 5,6,7-Trihydroxy-2-(4-hydroxyphenyl)-4H-chromen-4-one | |
| 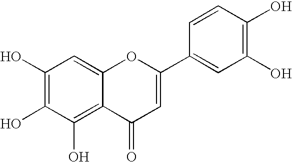 | Phytochemistry; 27; 1; 1988; 255-258. |
| 5,6,7-Trihydroxy-2-(3,4-dihydroxyphenyl)-4H-chromen-4-one | |
| 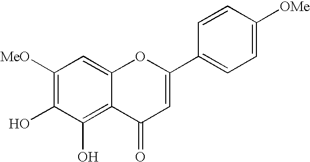 | Phytochemistry; 50; 7; 1999; 1189-1194. |
| 5,6-Dihydroxy-7-methoxy-2-(4-methoxyphenyl)-4H-chromen-4-one | |
| 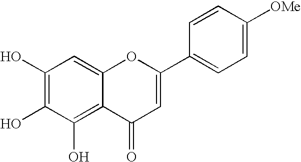 | Phytochemistry; 34; 1; 1993; 167-170. |
| 5,6,7-Trihydroxy-2-(4-methoxyphenyl)-4H-chromen-4-one | |
| 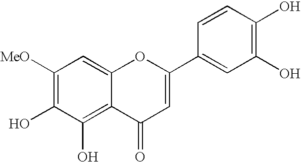 | Phytochemistry; 56; 8; 2001; 853-856. |
| 5,6-Dihydroxy-2-(3,4-dihydroxyphenyl)-7-methoxy-4H-chromen-4-one | |
| 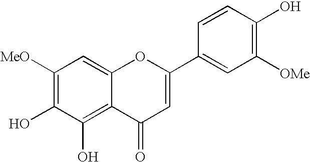 | Phytochemistry; 56; 6; 2001; 559-568. |
| 5,6-Dihydroxy-2-(4-hydroxy-3-methoxyphenyl)-7-methoxy- | |
| 4H-chromen-4-one | |
| 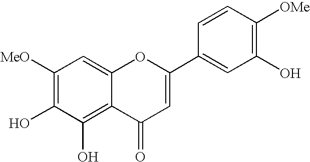 | Phytochemistry; 16; 1977; 1618. |
| 5,6-Dihydroxy-2-(3-hydroxy-4-methoxyphenyl)-7-methoxy- | |
| 4H-chromen-4-one | |
| 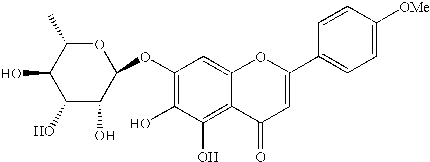 | Phytochemistry; 10; 1971; 2850. |
| 7-((2S,3R,4R,5R,6S)-tetrahydro-3,4,5-trihydroxy-6-methyl-2H- | |
| pyran-2-yloxy)-5,6-dihydroxy-2-(4-methoxyphenyl)-4H- | |
| chromen-4-one | |
| 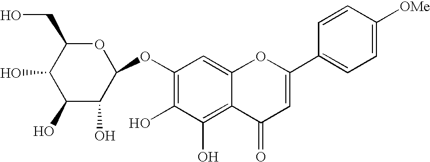 | Chem.Nat.Compd.(Engl. Transl.); 7; 1971; 563; |
| 7-((2S,3R,4S,5S,6R)-tetrahydro-3,4,5-trihydroxy-6-(hydroxy | |
| methyl)-2H-pyran-2-yloxy)-5,6-dihydroxy-2- | |
| (4-methoxyphenyl)-4H-chromen-4-one | |
| 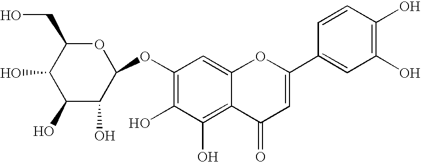 | Phytochemistry; 55; 3; 2000; 263 |
| 7-((2S,3R,4S,5S,6R)-tetrahydro-3,4,5-trihydroxy-6-(hydroxy | |
| methyl)-2H-pyran-2-yloxy)-5,6-dihydroxy-2-(3,4-dihydroxy | |
| phenyl)-4H-chromen-4-one | |
| 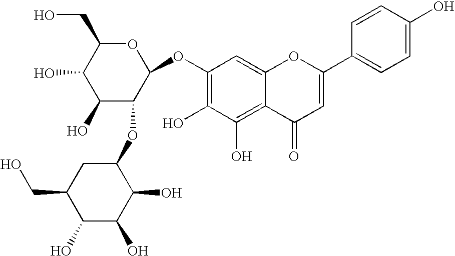 | Chem.Nat.Compd.(Engl. Transl.); 18; 2; 1982; 170-172; |
| 7-[4,5-dihydroxy-6-hydroxymethyl-3-(3,4,5-trihydroxy-6- | |
| hydroxymethyl-tetrahydro-pyran-2-yloxy)-tetrahydro-pyran- | |
| 2-yloxy]-5,6-dihydroxy-2-(4-hydroxy-phenyl)-chromen-4-one | |
| 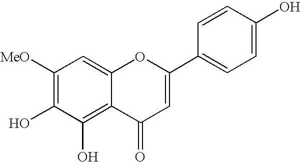 | Phytochemistry; 56; 6; 2001; 559-568. |
| 5,6-Dihydroxy-2-(4-hydroxyphenyl)-7-methoxy-4H-chromen-4-one | |
| 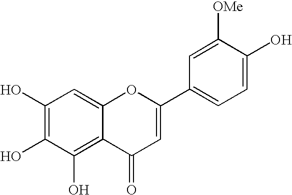 | Phytochemistry; 48; 6; 1998; 991-994. |
| 5,6,7-Trihydroxy-2-(4-hydroxy-3-methoxyphenyl)-4H-chromen-4-one | |
| 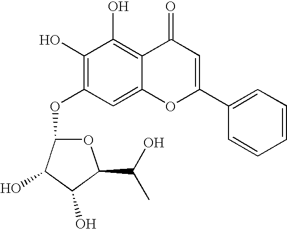 | Chem.Nat.Compd.(Engl. Transl.); 8; 1972; 239; |
| 7-((2S,3S,4R,5S)-tetrahydro-3,4-dihydroxy-5-((R)-1- | |
| hydroxyethyl)furan-2-yloxy)-5,6-dihydroxy-2-phenyl-4H-chromen-4-one | |
| 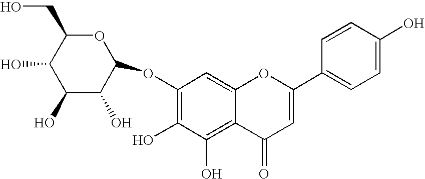 | Phytochemistry; 25; 9; 1986; 2135-2154. |
| 7-((2S,3R,4S,5S,6R)-tetrahydro-3,4,5-trihydroxy-6-(hydroxymethyl)- | |
| 2H-pyran-2-yloxy)-5,6-dihydroxy-2-(4-hydroxyphenyl)-4H-chromen-4-one | |
| 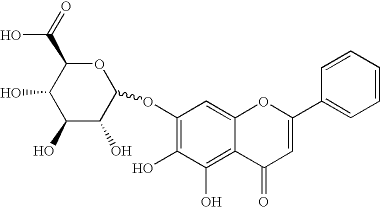 | Chem.Nat.Compd.(Engl. Transl.); 11; 1975; 106; |
| (2S,3S,4S,5R)-6-(5,6-dihydroxy-4-oxo-2-phenyl-4H-chromen- | |
| 7-yloxy)-tetrahydro-3,4,5-trihydroxy-2H-pyran-2-carboxylic acid | |
| 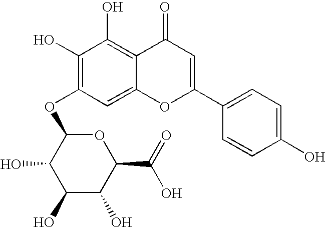 | Chem.Nat.Compd.(Engl. Transl.); 8; 1972; 385; |
| (2R,3R,4R,5S,6R)-6-(5,6-dihydroxy-2-(4-hydroxyphenyl)-4- | |
| oxo-4H-chromen-7-yloxy)-tetrahydro-3,4,5-trihydroxy-2H- | |
| pyran-2-carboxylic acid | |
| 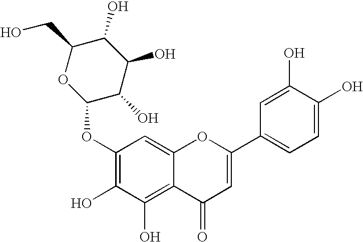 | Phytochemistry; 6; 1967; 1643, 1650. |
| 7-((2S,3S,4R,5R,6S)-tetrahydro-3,4,5-trihydroxy-6-(hydroxy | |
| methyl)-2H-pyran-2-yloxy)-5,6-dihydroxy-2-(3 ,4-dihydroxy | |
| phenyl)-4H-chromen-4-one | |
| 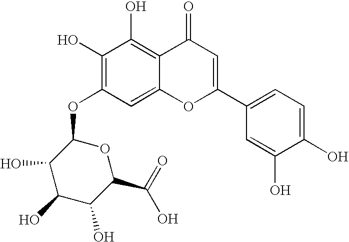 | Chem.Nat.Compd.(Engl. Transl.); 11; 1975; 106; |
| (2R,3R,4R,5S,6R)-6-(5,6-dihydroxy-2-(3,4-dihydroxyphenyl)- | |
| 4-oxo-4H-chromen-7-yloxy)-tetrahydro-3,4,5-trihydroxy- | |
| 2H-pyran-2-carboxylic acid | |
| 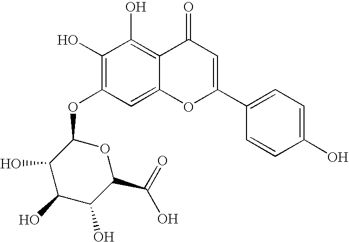 | Chem.Nat.Compd.(Engl. Transl.); 11; 1975; 106; |
| (2R,3R,4R,5S,6R)-6-(5,6-dihydroxy-2-(4-hydroxyphenyl)-4- | |
| oxo-4H-chromen-7-yloxy)-tetrahydro-3,4,5-trihydroxy-2H- | |
| pyran-2-carboxylic acid | |
| 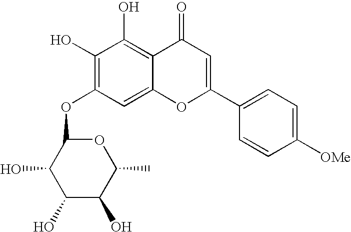 | Phytochemistry; 10; 1971; 2850. |
| 7-((2S,3S,4S,5S,6R)-tetrahydro-3,4,5-trihydroxy-6-methyl-2H- | |
| pyran-2-yloxy)-5,6-dihydroxy-2-(4-methoxyphenyl)-4H- | |
| chromen-4-one | |
| 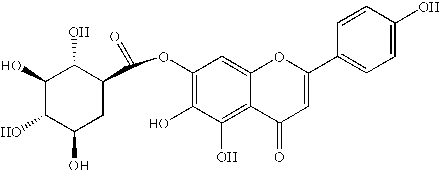 | Curr.Sci.; 48; 1979; 67. |
| (2S,3S,4S,5R,6R)-5,6-dihydroxy-2-(4-hydroxyphenyl)-4-oxo- | |
| 4H-chromen-7-yl-tetrahydro-3,4,5,6-tetrahydroxy-2H- | |
| pyran-2-carboxylate | |
| 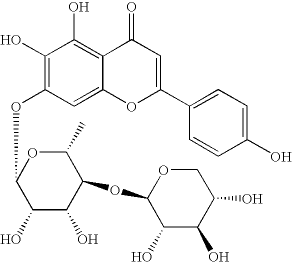 | Chem.Nat.Compd.(Engl. Transl.); 5; 1969; 423; |
| 7-[3,4-dihydroxy-6-methyl-5-(3,4,5-trihydroxy-tetrahydro- | |
| pyran-2-yloxy)-tetrahydro-pyran-2-yloxy]-5,6-dihydroxy-2- | |
| (4-hydroxyphenyl)-chromen-4-one | |
| 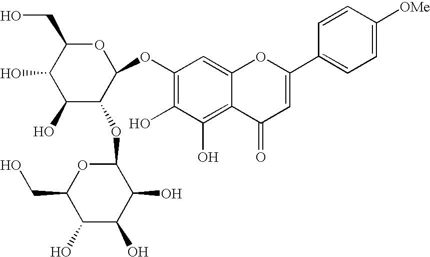 | Chem.Nat.Compd.(Engl. Transl.); 7; 1971; 563; |
| 7-[4,5-dihydroxy-6-hydroxymethyl-3-(3,4,5-trihydroxy-6- | |
| hydroxymethyl-tetrahydro-pyran-2-yloxy)-tetrahydro-pyran- | |
| 2-yloxy]-5,6-dihydroxy-2-(4-methoxy-phenyl)-chromen-4-one | |
| 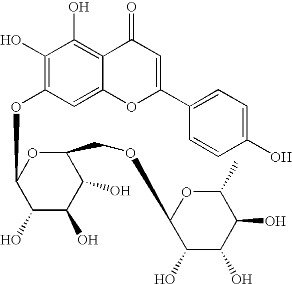 | Chem.Nat.Compd.(Engl. Transl.); 11; 1975; 444; |
| 5,6-dihydroxy-2-(4-hydroxy-phenyl)-7-[3,4,5-trihydroxy-6- | |
| (3,4,5-trihydroxy-6-methyl-tetrahydro-pyran-2-yloxymethyl)- | |
| tetrahydropyran-2-yloxy]-chromen-4-one | |
| 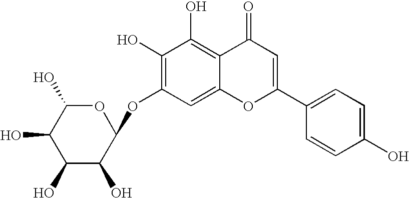 | Chem.Nat.Compd.(Engl. Transl.); 5; 1969; 516; |
| 7-((2R,3S,4S,5R,6R)-tetrahydro-3,4,5,6-tetrahydroxy-2H-pyran- | |
| 2-yloxy)-5,6-dihydroxy-2-(4-hydroxyphenyl)-4H-chromen-4-one | |
| 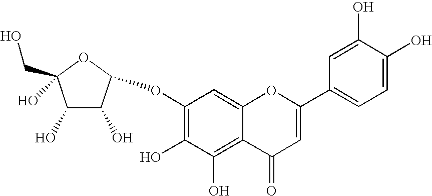 | Indian J.Chem. Sect.B; 19; 9; 1980; 822. |
| 7-((2S,3R,4S,5R)-tetrahydro-3,4,5-trihydroxy-5-(hydroxymethyl)furan- | |
| 2-yloxy)-5,6-dihydroxy-2-(3,4-dihydroxyphenyl)-4H-chromen-4-one | |
| 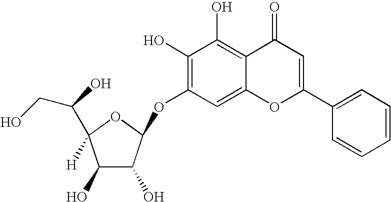 | Chem.Nat.Compd.(Engl.Transl.); EN; 26; 4; 1990; 464; |
| 7-((2S,3R,4R,5R)-tetrahydro-3,4-dihydroxy-5-((R)-1,2- | |
| dihydroxyethyl)furan-2-yloxy)-5,6-dihydroxy-2-phenyl-4H-chromen-4-one | |
| 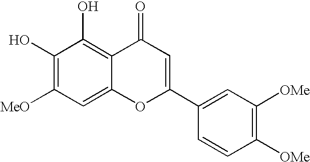 | Phytochemistry; 56; 6; 2001; 559-568. |
| 5,6-dihydroxy-7-methoxy-2-(3,4-dimethoxyphenyl)-4H-chromen-4-one | |
| 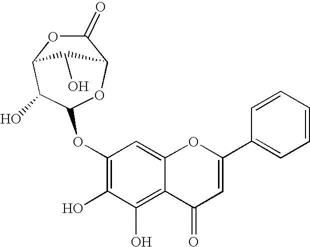 | Chem.Pharm.Bull.; 33; 6; 1985; 2411-2415. |
| 7-(4,8-dihydroxy-7-oxo-2,6-dioxa-bicyclo[3.2.1]oct-3-yloxy)- | |
| 5,6-dihydroxy-2-phenylchromen-4-one | |
| 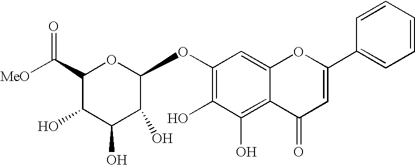 | Phytochemistry; 52; 5; 1999; 885-890. |
| (2S,3S,4S,5R,6S)-methyl-6-(5,6-dihydroxy-4-oxo-2-phenyl- | |
| 4H-chromen-7-yloxy)-tetrahydro-3,4,5-trihydroxy-2H-pyran- | |
| 2-carboxylate | |
| 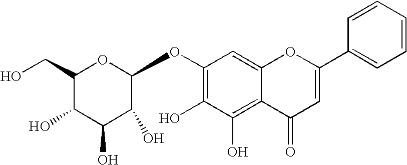 | Chem.Nat.Gompd.(Engl.Transl.); EN; 37; 5; 2001; 428-430; |
| 7-((2S,3R,4S,5S,6R)-tetrahydro-3,4,5-trihydroxy-6-(hydroxymethyl)- | |
| 2H-pyran-2-yloxy)-5,6-dihydroxy-2-phenyl-4H-chromen-4-one | |
| 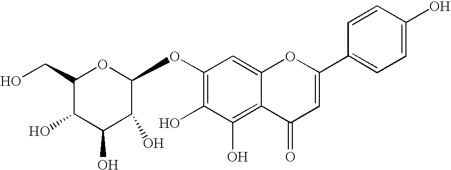 | Phytochemistry; 34; 1; 1993; 211-218. |
| 7-((2S,3R,4S,5S,6R)-tetrahydro-3,4,5-trihydroxy-6-(hydroxymethyl)- | |
| 2H-pyran-2-yloxy)-5,6-dihydroxy-2-(4-hydroxyphenyl)-4H-chromen-4-one | |
| 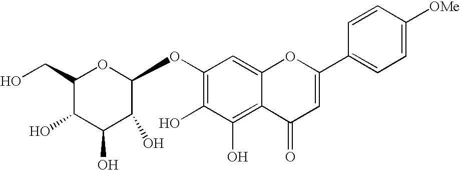 | Pharmazie; 41; 1; 1986; |
| 7-((2S,3R,4S,5S,6R)-tetrahydro-3,4,5-trihydroxy-6-(hydroxymethyl)- | |
| 2H-pyran-2-yloxy)-5,6-dihydroxy-2-(4-methoxyphenyl)-4H-chromen-4-one | |
| 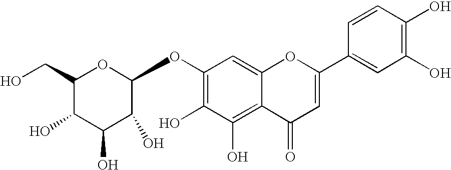 | Phytochemistry; 51; 3; 1999; 417-424. |
| 7-((2S,3R,4S,5S,6R)-tetrahydro-3,4,5-trihydroxy-6-(hydroxy- | |
| methyl)-2H-pyran-2-yloxy)-5,6-dihydroxy-2-(3,4-dihydroxy- | |
| phenyl)-4H-chromen-4-one | |
| 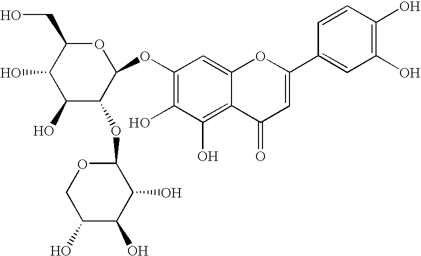 | Phytochemistry; 52; 6; 1999; 1165-1168. |
| 7-[4,5-dihydroxy-6-hydroxymethyl-3-(3,4,5-trihydroxy-tetra- | |
| hydropyran-2-yloxy)-tetrahydro-pyran-2-yloxy]-2-(3,4- | |
| dihydroxy-phenyl)-5,6-dihydroxy-chromen-4-one | |
| 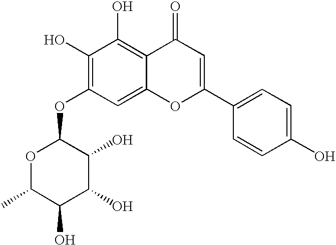 | Z.Naturforsch.B; GE; 42; 10; 1987; 1361-1364. |
| 7-((2S,3R,4R,5R,6S)-tetrahydro-3,4,5-trihydroxy-6-methyl-2H- | |
| pyran-2-yloxy)-5,6-dihydroxy-2-(4-hydroxyphenyl)-4H-chromen-4-one | |
| 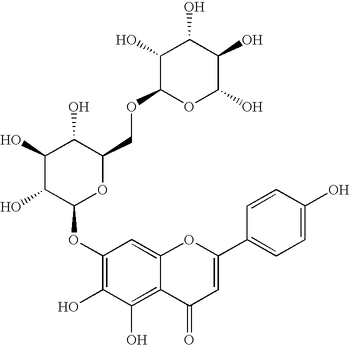 | Phytochemistry; 25; 9; 1986; 2135-2154. |
| 2-(3,4-dihydroxy-phenyl)-5,6-dihydroxy-7-[3,4,5-trihydroxy- | |
| 6-(3,4,5-trihydroxy-6-methyl-tetrahydro-pyran-2-yloxymethyl)- | |
| tetrahydropyran-2-yloxy]-chromen-4-one | |
| 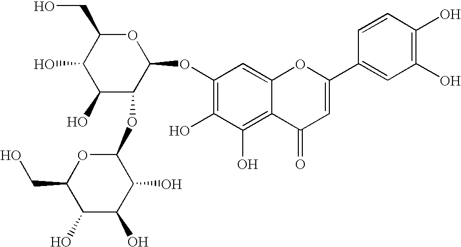 | Phytochemistry; 27; 1; 1988; 255-258. |
| 7-[4,5-dihydroxy-6-hydroxymethyl-3-(3,4,5-trihydroxy-6- | |
| hydroxymethyl-tetrahydro-pyran-2-yloxy)-tetrahydro-pyran- | |
| 2-yloxy]-2-(3,4-dihydroxy-phenyl)-5,6-dihydroxy-chromen-4-one | |
| 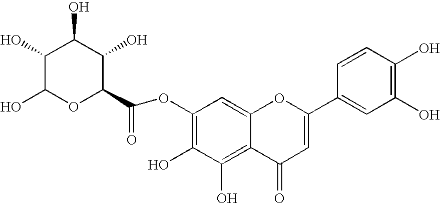 | Phytochemistry; 34; 1; 1993; 205-210. |
| (2S,3S,4S,5R)-5,6-dihydroxy-2-(3,4-dihydroxyphenyl)-4-oxo- | |
| 4H-chromen-7-yl-tetrahydro-3,4,5,6-tetrahydroxy-2H-pyran-2-carboxylate | |
| 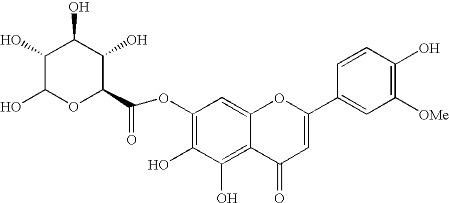 | Phytochemistry; 34; 1; 1993; 205-210. |
| (2S,3S,4S,5R)-5,6-dihydroxy-2-(4-hydroxy-3-methoxyphenyl)- | |
| 4-oxo-4H-chromen-7-yl-tetrahydro-3,4,5,6-tetrahydroxy- | |
| 2H-pyran-2-carboxylate | |
| 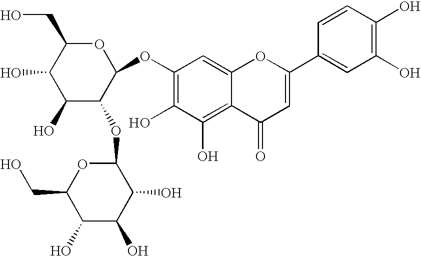 | Phytochemistry; 34; 1; 1993; 205-210. |
| 7-[4,5-dihydroxy-6-hydroxymethyl-3-(3,4,5-trihydroxy-6- | |
| hydroxymethyl-tetrahydro-pyran-2-yloxy)-tetrahydro-pyran- | |
| 2-yloxy]-2-(3,4-dihydroxy-phenyl)-5,6-dihydroxy-chromen-4-one | |
| 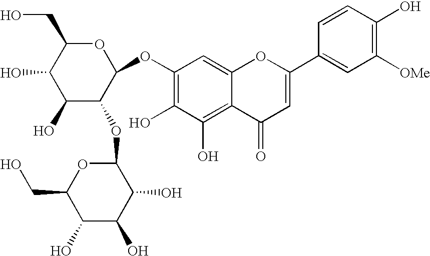 | Phytochemistry; 34; 1; 1993; 205-210. |
| 7-[4,5-dihydroxy-6-hydroxymethyl-3-(3,4,5-trihydroxy-6- | |
| hydroxymethyl-tetrahydro-pyran-2-yloxy)-tetrahydro-pyran- | |
| 2-yloxy]-2-(3-methoxy-4-hydroxy- | |
| phenyl)-5,6-dihydroxy-chromen-4-one | |
| 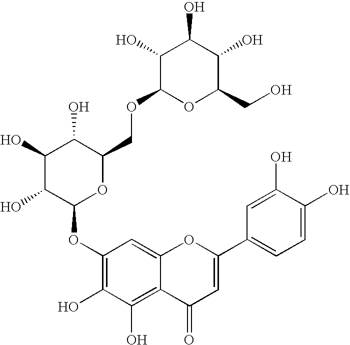 | Phytochemistry; 23; 4; 1984; 787-790. |
| 2-(3,4-dihydroxy-phenyl)-5,6-dihydroxy-7-[3,4,5-trihydroxy- | |
| 6-(3,4,5-trihydroxy-6-hydroxymethyl-tetrahydro-pyran-2- | |
| yloxymethyl)-tetrahydro-pyran-2-yloxy]-chromen-4-one | |
| 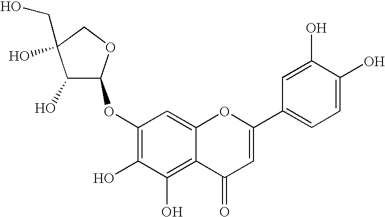 | Phytochemistry; 19; 1980; 2505-2506. |
| 7-((2S,3R,4R)-tetrahydro-3,4-dihydroxy-4-(hydroxymethyl)furan- | |
| 2-yloxy)-5,6-dihydroxy-2-(3,4-dihydroxyphenyl)-4H-chromen-4-one | |
| 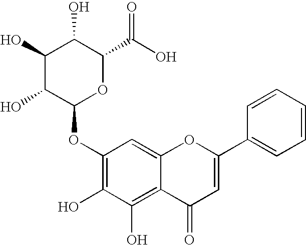 | Phytochemistry; 44; 1; 1997; 83-88. |
| (2R,3R,4S,5R,6S)-6-(5,6-dihydroxy-4-oxo-2-phenyl-4H-chromen- | |
| 7-yloxy)-tetrahydro-3,4,5-trihydroxy-2H-pyran-2-carboxylic acid | |
| 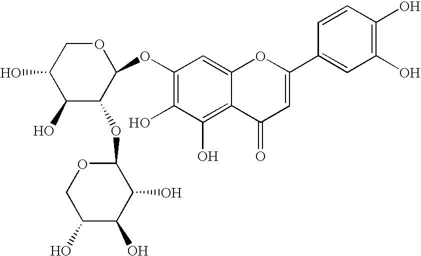 | Phytochemistry; 52; 6; 1999; 1165-1168. |
| 2-(3,4-dihydroxy-phenyl)-7-[4,5-dihydroxy-3-(3,4,5- | |
| trihydroxytetrahydro-pyran-2-yloxy)-tetrahydro-pyran-2-yloxy]- | |
| 5,6-dihydroxychromen-4-one | |
| 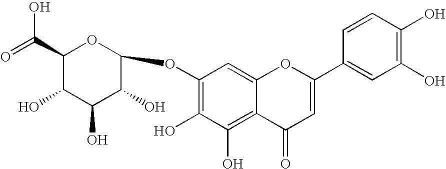 | Phytochemistry; 53; 8; 2000; 965-970. |
| (2S,3S,4S,5R,6S)-6-(5,6-dihydroxy-2-(3,4-dihydroxyphenyl)- | |
| 4-oxo-4H-chromen-7-yloxy)-tetrahydro-3,4,5-trihydroxy-2H- | |
| pyran-2-carboxylic acid | |
| 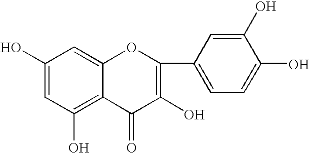 | Can be isolated from cassiae pulpa. |
| 3,5,7-Trihydroxy-2-(3,4-dihydroxyphenyl)-4H-chromen-4-one | |
| 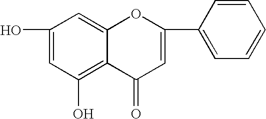 | Can be isolated from alamo. |
| 5,7-Dihydroxy-2-phenyl-4H-chromen-4-one | |
| 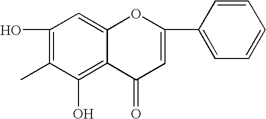 | Can be isolated from Pinus strobus |
| 5,7-Dihydroxy-6-methyl-2-phenyl-4H-chromen-4-one | |
| 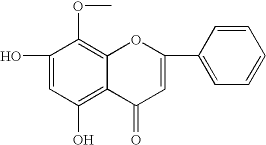 | Can be isolated from Radix Scutellariae. |
| 5,7-Dihydroxy-8-methoxy-2-phenyl-4H-chromen-4-one | |
| 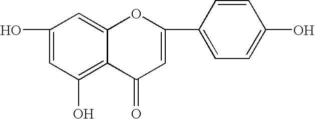 | Can be isolated from celery. |
| 5,7-Dihydroxy-2-(4-hydroxyphenyl)-4H-chromen-4-one | |
| 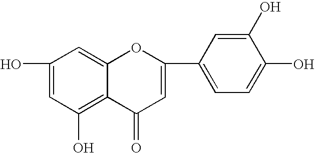 | Can be isolated from honeysuckle. |
| 5,7-Dihydroxy-2-(3,4-dihydroxyphenyl)-4H-chromen-4-one | |
| 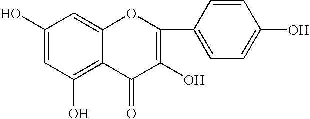 | Can be isolated from cascara. |
| 3,5,7-Trihydroxy-2-(4-hydroxyphenyl)-4H-chromen-4-one | |
| 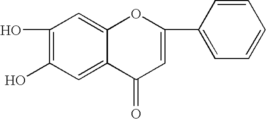 | J. Chem. Soc. 1933; 1073-1075 |
| 6,7-Dihydroxy-2-phenyl-4H-chromen-4-one | |
| 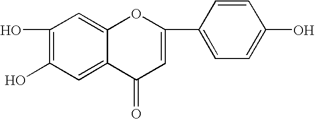 | Gazz. Chim. Ital.; 57; 1927. 607 |
| 6,7-Dihydroxy-2-(4-hydroxyphenyl)-4H-chromen-4-one | |
| 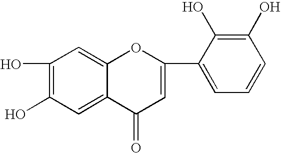 | Indian. J. Chem. Sect. B.; 21; 1982; 838-841 |
| 6,7-Dihydroxy-2-(2,3-dihydroxyphenyl)-4H-chromen-4-one | |
| 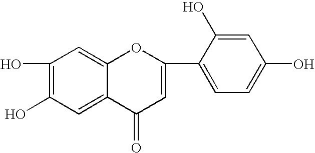 | Indian. J. Chem. Sect. B.; 21; 1982; 838-841 |
| 6,7-Dihydroxy-2-(2,4-dihydroxyphenyl)-4H-chromen-4-one | |
| 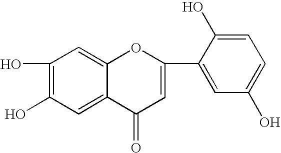 | Indian. J. Chem. Sect. B.; 21; 1982; 838-841 |
| 6,7-Dihydroxy-2-(2,5-dihydroxyphenyl)-4H-chromen-4-one | |
| 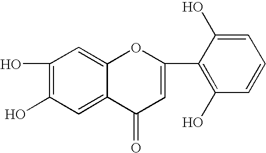 | Indian. J. Chem. Sect. B.; 21; 1982; 838-841 |
| 6,7-Dihydroxy-2-(2,6-dihydroxyphenyl)-4H-chromen-4-one | |
| 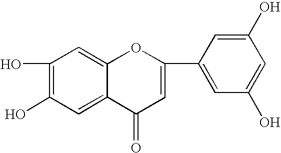 | Indian. J. Chem. Sect. B.; 21; 1982; 838-841 |
| 6,7-Dihydroxy-2-(3,5-dihydroxyphenyl)-4H-chromen-4-one | |
| 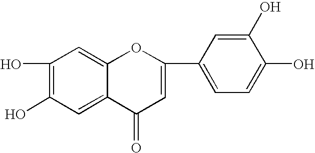 | Indian. J. Chem. Sect. B.; 21; 1982; 838-841 |
| 6,7-Dihydroxy-2-(3,4-dihydroxyphenyl)-4H-chromen-4-one | |

Those of skill in the art will appreciate that the compounds of the invention described herein may include functional groups that can be masked to create prodrugs. Such prodrugs are usually, but need not be, pharmacologically inactive until converted into their active drug form. In the prodrugs of the invention, any available functional moiety may be masked with a functional group to yield a prodrug. Myriad of functional groups that are cleavable under the desired conditions of use are known in the art. Thus, “prodrug” refers to a derivative of an active compound (drug) that undergoes a transformation under the conditions of use, such as within the body, to release an active drug. Prodrugs are frequently, but not necessarily, pharmacologically inactive until converted into the active drug. The cleavage to the active compound may proceed spontaneously, such as by way of a hydrolysis reaction, or it may be catalyzed or induced by another agent, such as by an enzyme, by light, by acid, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

A wide variety of functional groups suitable for masking functional groups in active compounds to yield prodrugs are well-known in the art. For example, a hydroxyl functional group may be masked as a sulfonate, ester or carbonate promoiety, which may be hydrolyzed in vitro to provide the hydroxyl group. An amino functional group may be masked as an amide, imine, phosphinyl, phosphonyl, phosphoryl or sulfenyl promoiety, which may be hydrolyzed in vivo to provide the amino group. A carboxyl group may be masked as an ester (including silyl esters and thioesters), amide or hydrazide promoiety, which may be hydrolyzed in vivo to provide the carboxyl group. Other specific examples of suitable progroups and their respective promoieties will be apparent to those of skill in the art.

6.2 Methods of Preparation

The flavonoids and the flavonoid-containing compounds of the invention can be extracted as described in the publications above, or they can be synthesized. The compounds of the present invention, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTY 3^{rd} Ed., Vols. A and B (Plenum 1992), and Green and Wuts, PROTECTIVE GROUPS IN ORGANIC SYNTHESIS 2^{nd} Ed. (Wiley 1991). Starting materials for the compounds of the invention may be obtained using standard techniques and commercially available precursor materials, such as those available from Aldrich Chemical Co., Sigma Chemical Co, Lancaster Synthesis (Windham, N.H.), Apin Chemicals, Ltd. (New Brunswick, N.J.), Ryan Scientifi'c (Columbia, S.C.), and Maybridge. Starting materials useful for preparing compounds of the invention and intermediates thereof are commercially available or can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995).

The procedures described herein for synthesizing the compounds of the invention may include one or more steps of protection and deprotection (e.g., the formation and removal of acetal and other protecting groups). Examples of protecting groups can be found in Greene and Wuts, Protective Groups in Organic Chemistry, 3^{rd} Ed., 1999, John Wiley & Sons, NY and Harrison et al., Compendium ofSynthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (e.g., methyl and ethyl esters, acetate or propionate groups or glycol esters) or alkylated such as benzyl and trityl ethers, as well as alkyl ethers, tetrahydropyranyl ethers, trialkylsilyl ethers (e.g., TMS or TIPPS groups) and allyl ethers.

In addition, the synthetic procedures disclosed below can include various purifications, such as column chromatography, flash chromatography, thin-layer chromatography (TLC), recrystallization, distillation, high-pressure liquid chromatography (HPLC) and the like. Also, various techniques well known in the chemical arts for the identification and quantification of chemical reaction products, such as proton and carbon-13 nuclear magnetic resonance (¹H and ¹³C NMR), infrared and ultraviolet spectroscopy (IR and UV), X-ray crystallography, elemental analysis (EA), HPLC and mass spectroscopy (MS) can be used as well. Methods of protection and deprotection, purification and identification and quantification are well known in the chemical arts.

6.3. Therapeutic Use of the Compounds of the Invention

The “anti-fibrotic” activity described herein is different from “fibrinolytic” or “anti-fibrin” activity. The “fibrinolytic” or “anti-fibrin” activity refers to the biological ability of a pharmaceutical substance to (1) prevent fibrin formation (formation of a blood clot) or (2) lyses a formed blood clot.

The “anti-fibrotic” activity discovered by the present inventor and as used herein refers to the ability of an active compound to (1) prevent an excessive pathologic accumulation of collagenous scar or connective tissue in various body structures and organs (usually triggered by some injury, allergy, infection, or by some inherited genetic aberration), or (2) cause the non-surgical removal or biological dissolution of an existing excessive and pathologic accumulation of fibrotic collagenous tissue (for example, as in the dissolution of life-threatening fibrotic lesions of the lung found in patients with asbestosis).

6.3.1. Connective Tissue Proteins of Mammals

Three major classifications of connective tissue proteins are recognized with the largest portions consisting of collagen types (70 to 80%) and elastin types (15 to 20%). A miscellaneous group constitutes the third and smallest class.

The general biochemical characteristics of the collagen types that constitute the principal protein (1) in normal white connective tissue and (2) in scar or fibrotic tissue, as contrasted with elastin types. For example, collagen is sparingly soluble in water, but readily converted to water-soluble gelatin upon boiling in an acid or alkali. In contrast, the highly water-soluble elastin does not convert to gelatin upon boiling in an acid or alkali.

The elastin constitutes the principal protein of yellow connective tissue found in elastic structures such as the walls of larger blood vessels and walls of lung alveoli.

Investigations on the molecular biochemical level of tissues have demonstrated a very slow turnover rate for metabolic processes involving fibrotic lung collagen. In fact, the metabolic rate is measured in years. By contrast, the metabolic rates of the other connective tissue collagens including elastin and the like are measured and expressed in hours and days (White, Handler, and Smith, 1973, page 983).

6.3.2. Interstitial Proliferation (Hyperplasia) of Fibroblast-type Cells in Lungs and other Organ Tissues

The synthesis of various collagens found in scar or fibrotic structures takes place in fibroblast cells, which then extrude the collagen into the surrounding matrix. During this wound repair process, there are (1) a rapid proliferation and increase in the number of fibroblasts at the site, and (2) a sharp rise in the rate of the synthesis and extrusion of collagen. If these two phenomena are not prevented, the pathologic and progressive accumulation of collagen would cause polymerization and fibrotic disease (for example, impairment of respiratory function, impaired circulatory function via fibrotic changes in arterial walls, fibrotic degeneration of renal and liver function, degenerative musculoskeletal function, fibrotic degeneration of cardiac muscle or skeletal muscle, fibrotic degenerative changes in neuronal tissues in the central nervous system as well as the peripheral nervous system, etc.). [S. L. Robbins, R. S. Contrans, V. Kumar, “Pathologic Basis of Disease”, 6^{th} edition, pages 40-84, Saunders, Philadelphia, Pa. (Pub.)].

With pulmonary interstitial fibrotic hyperplasia, small and firm nodules are palpable throughout the lung tissue, and upon gross examination are recognized from their opaque, airless structure to be foci of abnormal accumulations of fibrotic connective tissue. Such foci vary in size and color according to their age. Their aggressive and continued enlargement and coalescence ultimately leads to collagenous solidification of large segments of the lungs.

These enlarging foci also impinge upon the lung capillaries thereby to reduce pulmonary blood flow, and at the same times, impede lymphatic drainage from the lungs. As a consequence, exudates accumulate within the alveoli, and secondary thickening of the alveolar wall ensues. These interacting processes sharply reduce the efficiency of the gaseous exchange in the lung alveoli, which is a primary function of the normal lung.

In addition, these pulmonary fibrotic alternations and accumulations raise the pulmonary blood vessel resistance and lead to sharply elevate pulmonary blood pressure. Prolonged elevated pulmonary blood pressure almost invariably leads to congestive heart failure in addition to the cyanosis caused by inadequate pulmonary exchange of oxygen and carbon dioxide. The prognosis is poor and the incidence of severe morbidity and deaths is high.

Furthermore, the fibrosis of the lung impairs the physiological and biochemical functions of the lung that are independent of the pulmonary gas exchange (oxygen and carbon dioxide) role of the lungs cited above. These include: filtration, degradation, and removal of the following substances:
(1) aged leucocytes from the blood, and
(2) particulate matter (for example, tissue cell debris, blood cell aggregates, inert foreign matter, small thrombi); and
(3) synthesis of adequate supplies of heparin.

Heparin is a useful substance that normally prevents the formation of life-threatening blood clots in the major blood vessels (for example, cerebral and coronary blood vessels).

6.3.3. Differentiation Between Anti-fibrotic Activity and Anti-inflammatory Activity

Pharmacological anti-fibrotic activity as exemplified by the arrest and removal of lung scarring (interstitial hyper-plasia and fibrotic foci), or pathologic fibrotic lesions in other organs and tissues described herein, is clearly distinct from and independent of any pharmacological anti-inflammatory activity.

The debilitating pathologic degeneration of organs and tissues affected by fibrotic disease continues for extended periods of time, measured in months or years, beyond the short-term (hours and days) of exacerbating inflammatory flare-ups (classical clinical and histophathological signs of edema, local heat, and leukocytic infiltration have disappeared).

6.3.4. Treatment and Prevention of Fibrotic Diseases

The compositions of this invention are effective for treatment of disease caused by the pathologic and excessive fibrotic accumulations that include pulmonary fibrosis, benign prostate hypertrophy, coronary infarcts, cerebral infarcts, myocardiac fibrosis, musculoskeletal fibrosis, post-surgical adhesions, liver cirrhosis, real fibrotic disease, fibrotic vascular disease (atherosclerosis, varix, or varicose veins), scleroderma, Alzheimer's disease, diabetic retinopathy, glaucoma, etc. The pulmonary fibrosis may have been chemically induced, for example, by the anti-cancer drugs bleomycin or cyclophosphamide. The compositions of this invention not only arrest the formation of new fibrotic tissue but causes removal of previously formed fibrotic collagen-containing tissue. These pharmacological properties were heretofore unknown.

The present invention arrests formation of or causes removal of a pathogenic accumulation of water-insoluble collagenous connective tissue (for example, excessive scar or lesional fibrotic tissue, etc.). By medicinally removing such pathologic collagenous tissue in fibrotic lungs, the invention eliminates or prevents:
(1) the mechanical compression or occlusion (stenosis) of blood vessels (for example, pulmonary arteries, veins, and capillaries), pulmonary bronchioles, and alveoli;
(2) the inhibition of the primary respiratory function of the alveoli of the lungs, namely, the exchange of oxygen and carbon dioxide gases; and
(3) the increased pulmonary blood vessel resistance (corpulmonale) which readily causes fatal congestive heart failure because of the excessive workload on cardiac muscle that is engendered by the corpulmonale.

The dramatic and novel pulmonary anti-fibrotic activity of balcalin and balcalein have been observed and demonstrated in animal experiments (rats).

The acute toxicity of the representative ingredients, balcalein and balcalin, in the present invention are about 11 g/kg and 14 g/kg body weight, respectively.

The anti-fibrotic effect in pulmonary fibrosis was demonstrated upon oral administration to rats in this invention.

6.3.5. Veterinary and Livestock Uses of the Compounds of the Invention

The compositions in the forms of compounds and extracts of the invention can be administered to a non-human animal for a veterinary use for treating or preventing a disease or disorder disclosed herein.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, or guinea pig. In another preferred embodiment, the non-human animal is a fowl species, most preferably a chicken, turkey, duck, goose, or quail.

6.3.6. Additional Livestock Uses of the Compounds of the Invention

In addition to veterinary uses, the compounds of the invention can be used to reduce the fat content of livestock to produce leaner meats. Alternatively, the compounds of the invention can be used to reduce the cholesterol content of eggs by administering the compounds to a chicken, quail, or duck hen. For non-human animal uses, the compounds of the invention can be administered via the animals' feed or orally as a drench composition.

6.3.7. Therapeutic/Prophylactic Administration and Compositions

Due to the activity of the compounds of the invention, the compounds are advantageously useful in veterinary and human medicine. As described in Section 5.2.4. above, the compounds and the extracts of the invention are useful for the treatment or prevention of pulmonary fibrosis, benign prostate hypertrophy, coronary infarcts, cerebral infarcts, myocardiac fibrosis, musculoskeletal fibrosis, post-surgical adhesions, liver cirrhosis, real fibrotic disease, fibrotic vascular disease (atherosclerosis, varix, or varicose veins), scleroderma, Alzheimer's disease, diabetic retinopathy, glaucoma, etc.

The invention provides methods of treatment and prophylaxis by administration to a patient of a therapeutically effective amount of a composition comprising a compound and the extract of the invention. The patient is an animal, including, but not limited, to an animal such a cow, horse, sheep, pig, chicken, turkey, quail, cat, dog, mouse, rat, rabbit, guinea pig, etc., and is more preferably a mammal, and most preferably a human.

The present compositions, which comprise one or more compounds or the extracts of the invention, are preferably administered orally. The compounds of the invention may also be administered by any other convenient route, for example, by infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds of the invention into the bloodstream.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In certain embodiments, for example, for the treatment of Alzheimer's Disease, it may be desirable to introduce one or more compounds of the invention into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compounds of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the compounds of the invention, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a compound of the invention, preferably in purified form, together with a suitable amount of a pharmaceutically acceptable vehicle so as to provide the form for proper administration to the patient.

In a specific embodiment, the term “pharmaceutically acceptable” means approved by a regulatory agency of the Chinese SFDA and FDA or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention and pharmaceutically acceptable vehicles are preferably sterile. Water is a preferred intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulation, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the compounds of the invention are formulated in accordance with routine procedures as a pharmaceutical composition adapted for intravenous administration to human beings. Typically, compounds of the invention for intravenous administration are solutions in sterile isotonic aqueous buffer. Where necessary, the compositions may also include a solubilizing agent. Compositions for intravenous administration may optionally include a local anesthetic such as lignocain to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 800 milligrams of a compound of the invention per kilogram body weight. In specific preferred embodiments of the invention, the oral dose is 0.01 milligram. to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In a most preferred embodiment, the oral dose is 20 milligrams of a compound of the invention per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and l milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and another lipid-mediating compound, including but not limited to a statin, a thiazolidinedione, or a fibrate.

6.4. Combination Therapy

In certain embodiments of the present invention, the compounds of the invention can be used in combination therapy with at least one other therapeutic agent. The compound of the invention and the therapeutic agent can act additively or, more preferably, synergistically. In a preferred embodiment, a composition comprising a compound of the invention is a administered concurrently with the administration of another therapeutic agent, which can be part of the same composition. In another embodiment, a composition comprising a compound of the invention is administered prior or subsequent to administration of another therapeutic agent. As many of the disorders for which the compounds of the invention are useful in treating are chronic disorders, in one embodiment combination therapy involves alternating between administering a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The present compositions can be administered together with a statin. Statins for use in combination with the compounds of the invention include but are not limited to atorvastatin, pravastatin, fluvastatin, lovastatin, simvastatin, and cerivastatin.

The present compositions can also be administered together with a Peroxisome Proliferator-Activated Receptor (PPAR) agonist, for example a thiazolodinedione or a fibrate. Thiaxolidinediones for use in combination with the compounds of the invention include but are not limited to 5-((4-(2-(methyl-2-pyridinylamino)ethoxy)phenyl)methyl)-2, 4-thiazolidinedione, troglitazone, pioglitazone, ciglitazone, WAY-120,744, englitazone, AD 5075, darglitazone, and rosiglitazone. Fibrates for use in combination with the compounds of the invention include but are not limited to gemfibrozil, fenofibrate, clofibrate, or ciprofibrate. As mentioned previously, a therapeutically effective amount of a fibrate or thiazolidinedion often has toxic side effects. Accordingly, in a preferred embodiment of the present invention, when a composition of the invention is administered in combination with a PPAR agonist, the dosage of the PPAR agonist is below that which is accompanied by toxic side effects.

### 7. EXAMPLE: METHOD OF ISOLATION OF BAICALIN

Dried, powdered root of Scultellaria baicalensis Leorgi (1000 g) was suspended in water (8 L) and heated to reflux for 1 hour. Cooled and filtered, the residue was suspended in water (6 L) and heated to reflux for 45 minutes. Cooled and filtered, the residue was again suspended in water (5 L) and heated to reflux for 30 minutes. Cooled and filtered, the combined filtrates was warmed to 80° C. and acidified with 12N HCl to pH=1.0. The mixture was kept at 80° C. for 1 hour then filtered. The solid was washed with alcohol (50% in water, v/v) to pH=7.0 and dried in vacuo to give the crude product (55 g).

The crude product (55 g) was suspended in absolute alcohol (2.5 L) and refluxed for 2 hours then filtered in vacuo while the mixture was hot. This process was repeated for two times. The solid was dissolved in DMF (350 mL) and filtered. Acetone (350 mL) was added to the filtrate then diluted with water (2 L). Filtered in vacuo, the solid was redissolved in absolute alcohol (350 mL) under ultrasonic then filtered in vacuo. The collected solid was redissolved in acetone (350 mL) under ultrasonic then filtered in vacuo and dried to give the product (31 g, purity >97% by HPLC).

### 8. EXAMPLE: METHOD OF PREPARATION OF BAICALIEN

Baicalin (120 g) was added to concentrated H₂SO₄ (500 mL) with vigorously stirring. Water (20 mL) was added to the reaction mixture after baicalin dissolved completely. The stirring was continued for 3 hours then the mixture was slowly poured into ice water (4 L) (keep the temperature below 50° C.). The mixture was filtered and the solid was washed with water. Recrystallization twice from acetone gave the produce (28 g). ¹H NMR (DMSO-d₆) δppm 12.66(1H, s), 10.61(1H, s), 8.84(1H, s), 8.02-8.12(2H, m), 7.56-7.64(3H, m), 6.93(1H, s), 6.64(1H, s).

### 9. EXAMPLE: METHOD OF PREPARATION OF MIXTURE (EXTRACTS) CONTAINING BAICALIN FROM Scultellaria baicalensis Leorgi

Dried, powdered root of Scultellaria baicalensis Leorgi (500 g) was suspended in water (5 L) and heated to reflux for 1 hour. The mixture was filtered and the solution was acidified to pH=1-2 with concentrated hydrochloric acid at 80° C. The mixture was kept at 80° C. for 30-60min then filtered. The solid was collected and dried in vacuo to give about 20 g of the extract that contains about 74% of baicalin.

### 10. EXMPLE: THE EFFICACY OF BAICALIN ON PULMONARY FIBROSIS IN BLEOMYCIN-INDUCED RATS

Material and methods:

Animals and Reagents: Male and female Sprague-Dawley rats weighing 200-250 grams were purchased from Shanghai Sippr-BK Lab. Animal Co. Ltd, Licence number SCXK (Shanghai) 2002-0006, Shanghai. The animals were housed four per cage and had access to water and laboratory chow ad libitum. Baicalin was isolated by Shanghai Comman Pharmaceutical Co., Ltd. (Shanghai). All other chemicals were reagent grade and obtained from standard commercial sources.

Treatment of Animals: the rats were acclimatized in a special room with constant temperature and filtered air flow one week prior to the start of the experiment. A 12 h light/dark cycle was maintained. The rats were fed ad libitum rat chow. The rats were randomized and sorted into three experimental groups, each group consists of 10 rats, 5 of males and 5 of females: (1) saline-instilled (SA) fed with saline; (2) Bleomycin-instilled (BL) fed with saline; and (3) Bleomyci-instilled and baicalin-treated (BT) via gavage, 450 mg/kg/day, once a day. Rats were anaesthetized with sodium pentobarbital (90-100 mg/kg, IP) and were intratracheally instilled with dose of 5 mg/kg in a volume of 2mL/kg once. The animals in the SA control group received an equivalent volume of sterile isotonic saline intratracheally in the same ways. Bleomycin was freshly dissolved in sterile isotonic saline before the instillation. Baicalin-treated rats were administered with baicalin, 450 mg/kg/day, via gavage once a day. The other two group were administered with same volume of saline, via gavage once a day

Animal Sacrifice and Tissue Processing: all rats were sacrificed using an overdose of sodium pentobarbital (120-125 mg/kg) at 28 days following the instillation of BL or saline.

Morphologic Evaluation: four rats from each group were used for morphological evaluation. After the trachea was cannulated, a thoracotomy was performed and the pulmonary vasculature was ligated at the base of the heart. Lungs were fixed by airway instillation with a cacodylate-buffered glutaraldehyde-paraformaldehyde fixative (400 mOsm, pH 7.0) at 30 cm of H₂O pressure lungs were fixed for at least 2 h, and blocks of tissue were cut from at least two sagittal slabs from the left, right cranial and right caudal lobes of each lung using the fractionator method to obtain a stratified sampling of lung tissue. Each slab was dehydrated in 95% ethanol and embedded in paraffin. Two sections from each lobe were cut and stained with hematoxylin and eosin. A lesion was defined as alveolitis consisting of thickened intra-alveolar septa resulting from edematous swelling, fibrosis or the presence of inflammatory cells in either interstitium or airways.

Processing of Lungs for Biochemical Assay: lungs of each animal designated for biochemical studies were perfused in situ through the right side of the heart with 20 mL ice-cold saline, and then quickly frozen in liquid nitrogen before storing at −80° C. Subsequently, the frozen lungs were thawed and homogenized in 0.1 M KCl, 0.02 M Tris HCl buffer (pH 7.6) with a Polytron homogenizer. After thoroughly mixing the homogenate, its total volume (10-12 mL) was recorded. Samples were divided into aliquots and stored at −80° C.

Preparation of lung homogenates: Liver samples were obtained at the moment of sacrifice and 150 mg were subjected to acid hydrolysis to determine the amount of total protein levels using Bradford assay of protein quantification [Bradford Anal Biochem 1976;72:248-254.].

Statistical Analysis of Data: the biochemical data are expressed on the basis of per lung. Expression of the data on a per lung basis avoids the artificial lowering of the values in BL-treated animals due to presence of proteins of extra-pulmonary origin. All values are reported as the mean ± standard error (SE). The data were compared within the three groups using two-way analysis of variance (SIGMASTAT) and Student-Newman-Keuls method. A value of p≦0.05 was considered the level of biological significance.

Results:

1. Total protein content, mononuclear cell counting and LDH activities from rat lung tissues in SA-, BL- and baicalin-treated groups. Total protein content, mononuclear cell counting and LDH activities from rat lung tissues in SA-, BL- and baicalin-treated groups are shown in table 3.

TABLE 3
Assays of Bronchoalveolar Lavage Fluid from
BL + Baicalin, BL and Vehicle treated groups

| | | Mononuclear | | |
|---|---|---|---|---|
| | | cells | | |
| Groups | n | (×10⁴/ml) | LDH (U/L) | Protein(g/L) |
| Vehicle | 10 | 60.0 ± 20.3 | 1219.54 ± 273.26 | 0.41 ± 0.16 |
| BL | 10 | 652.9 ± 344.7 | 2247.52 ± 608.33** | 2.22 ± 0.74** |
| BL + | 10 | 465.0 ± 320.2 | 1331.61 ± 516.68^{#} | 1.85 ± 0.52** |
| Baicalin | | | | |

**p < 0.01 vs. Vehicle
^{#}p < 0.05 vs. BL
Note:
LDH denotes Lactate Dehydrogenase.
BL denotes Bleomycin.

2. Histopathological examination for alveolitis and pulmonary fibrosis from lung tissues in SA-, BL- and baicalein-treated groups Histopathological examination for alveolitis and pulmonary fibrosis from lung tissues in SA-, BL- and baicalein-treated groups are.shown in table 4.

TABLE 4
Histopathological Examination in BL + Baicalin,
BL and Vehicle treated groups

| | | Alveolitis | | | | Pulmonary | | | |
|---|---|---|---|---|---|---|---|---|---|
| | | Score | | | | Fibrosis Score | | | |
| Groups | n | 0 | I | II | III | 0 | I | II | III |
| Vehicle | 10 | 10 | 0 | 0 | 0 | 10 | 0 | 0 | 0 |
| BL | 10 | 0 | 0 | 8 | 2 | 0 | 0 | 9 | 1 |
| BL + Baicalin | 10 | 0 | 7 | 3 | 0 | 0 | 6 | 4 | 0 |

X² test: There are statistically significant reduction (p < 0.01) in the amount of alveolitis and pulmonary fibrosis in rats receiving baicalin as compared to placebo (BL) control group.
Note:
BL denotes Bleomycin.

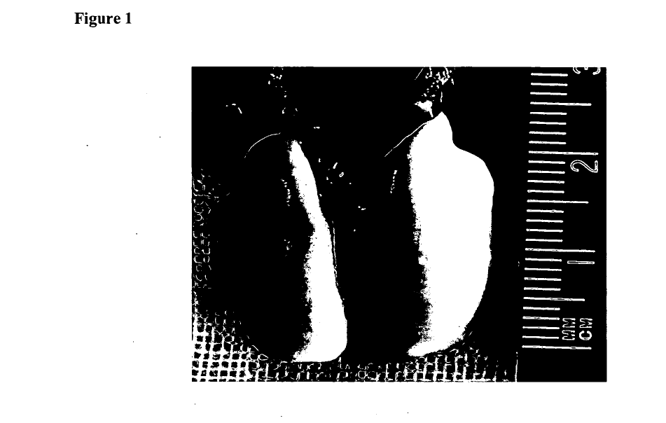

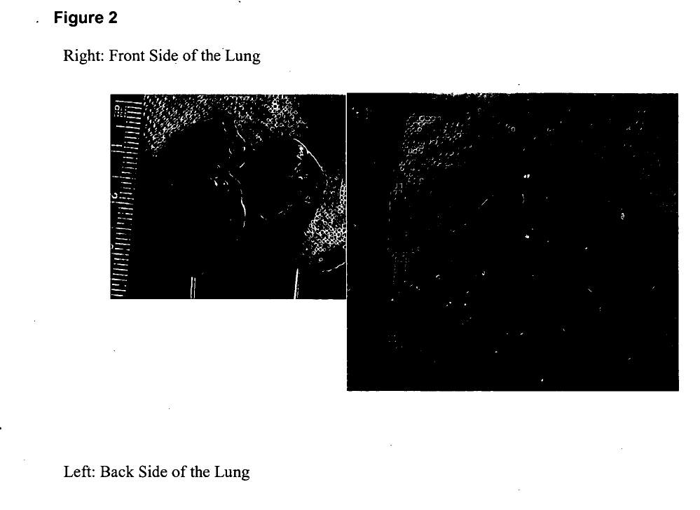

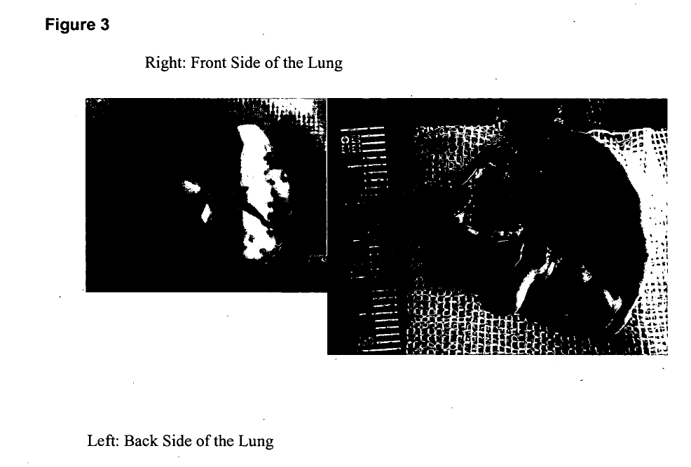

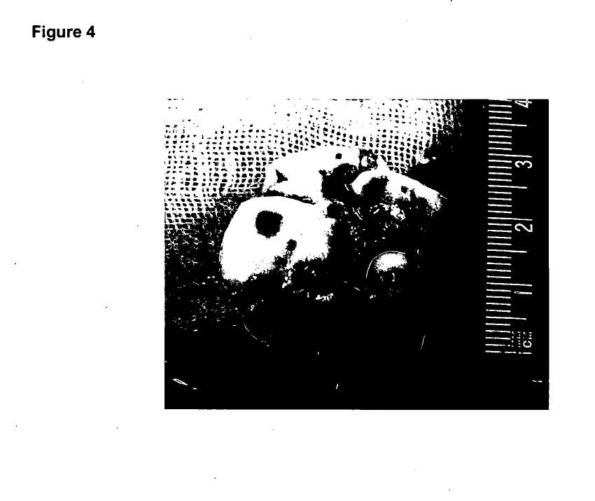

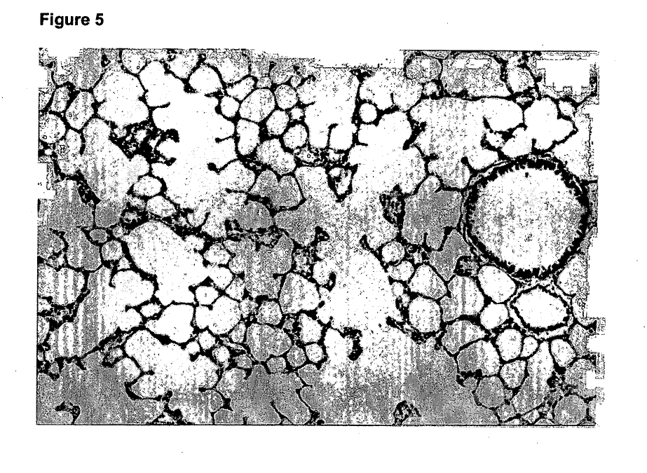

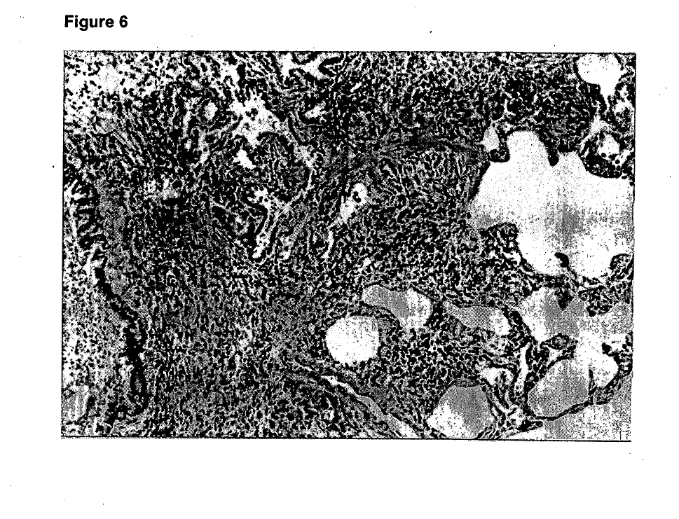

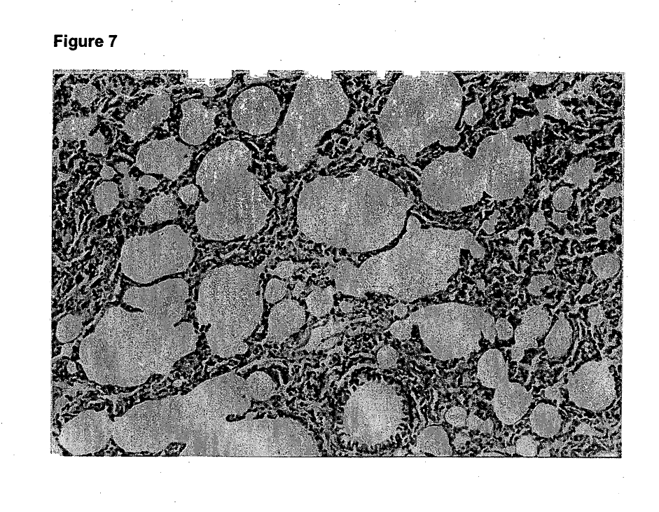

3. Morphology: Lungs from the control SA group showed no lesions and appeared normal in all aspects of their morphology (FIGS. 1 and 5). Lungs from the BL-treated rats in BL group (FIGS. 2 and 6) showed distinct differences as compared with the BT rats (FIGS. 3, 4 and 7) in BT group showing less severe lesions. The rats in the BL group showed multifocal lesions containing an accumulation of extracellular fibers and a cellular infiltrate of predominantly mononuclear cells. These lesions were mainly located in peribronchiolar, perivascular and the alveolar interstitium, and some lungs showed considerable mononuclear cell alveolitis. Lungs from the BT group showed lesions in the same locations but of a more mild nature than that of the BL group. The lesions in the lungs from the BT group had less extracellular fibers and some lobes showed a moderate degree of mononuclear cell alveolitis as compared with the BT rats.
4. Fibrotic index: A dramatic effect of oral baicalin was seen through an improvement in the remarkable difference in fibrotic index when data from single animals are tabled in table 2.
5. Toxicity: LD50 for baicalin is 11 g/kg.
   Discussion:

Despite many recent advances in the development of new forms of therapy, idiopathic pulmonary fibrosis still remains a highly lethal disease with a prognosis similar to that of lung cancer. Most of the drugs currently employed to treat this disease have debilitating systemic toxicity. While in vitro studies at the cellular and biochemical levels are essential in elucidating the mechanism of fibrosis, in vivo studies using reproducible animal models of lung fibrosis are still needed to develop and evaluate the efficacy of new compounds for their potential antifibrotic effects. We have consistently demonstrated that treatment with baicalin offers a marked protection against lung fibrosis in a single dose BL-rat model. Baicalin is orally effective since its antifibrotic effect is obtained by gavage, with very low toxicity (LD50=11 g/kg). The present study was carried out to find out whether or not baicalin would retard the progression of BL-induced lung fibrosis once it has already started. In order to answer this question, we employed a three-dose BL-rat model of lung fibrosis. In this model, baicalin was fed by gavage starting after the instillation of the BL and continued throughout the study. It is interesting that this regimen of baicalin treatment suppressed the BL-induced increases in the connective tissue and inflammation reactivity of the lungs in rats in the BT group as revealed biochemically and cellularly by reduced levels of lung total protein and reduced activities of lung LDH and mononuclear cell counting as compared to rats treated with BL in the BL group. In addition, the histopathological findings also support the biochemical findings in the sense that lungs from rats in the BT group had considerably less extracellular fibers and mononuclear cell alveolitis than the rats in the BL group that had multifocal lesions containing an accumulation of extracellular fibers and a cellular infiltrate of predominantly mononuclear cells.

Regardless of the mechanisms, the results of the present study indicate that oral intake of baicalin following the initiation of lung injury had beneficial effects by ameliorating lung fibrosis. This conclusion is based on the findings that baicalin treatment minimized the BL-induced lung inflammation and fibrosis in the BT group as evaluated by biochemical measurements of decreased total protein and mononuclear cell counting levels and decreased activities of LDH and by histopathological findings of reduced lung lesions. In view of these findings, it is tempting to speculate that baicalin a newly discovered compound has a great therapeutic potential for not only preventing, but also treating lung fibrosis in the initial stages of its development.

Each value represents the mean ±SEM of 10 animals. The abbreviations stand for: SA=saline installed; BL=Bleomycin-treated; BT=BL-instilled with Baicalin-treated. Student's T value was 2.43, with P less than 0.02 (highly significant statistically).

### 11. EXMPLE: THE EFFICACY OF BAICALEIN ON PULMONARY FIBROSIS IN BLEOMYCIN-INDUCED RATS

Material and methods:

Animals and Reagents: Male and female Sprague-Dawley rats weighing 200-250 grams were purchased from Shanghai Sippr-BK Lab. Animal Co. Ltd, Licence number SCXK (Shanghai) 2002-0006, Shanghai. The animals were housed four per cage and had access to water and laboratory chow ad libiturn. Baicalin was isolated by Shanghai Comman Pharmaceutical Co., Ltd. (Shanghai). All other chemicals were reagent grade and obtained from standard commercial sources.

Treatment of Animals: the rats were acclimatized in a special room with constant temperature and filtered air flow one week prior to the start of the experiment. The 12 h light/dark cycle was maintained. The rats were fed ad libiturn rat chow. The rats were randomized and sorted into three experimental groups, each group consists of 10 rats, 5 of males and 5 of females: (1) saline-instilled (SA) fed with saline; (2) Bleomycin-instilled (BL) fed with saline; and (3) Bleomycin-instilled and baicalein-treated (BT) by gavage, 350 mg/kg/day, once a day. Rats were anaesthetized with sodium pentobarbital (90-100 mg/kg, IP) and were intratracheally instilled with dose of Smg/kg in a volume of 2 mL/kg once. The animals in the SA control group received an equivalent volume of sterile isotonic saline intratracheally in the same ways. Bleomycin was freshly dissolved in sterile isotonic saline before the instillation. Baicalein-treated rats were administered with baicalein, 450 mg/kg/day, by gavage once a day. The other two group were administered with same volume of saline, by gavage once a day

Animal Sacrifice and Tissue Processing: all rats were sacrificed using an overdose of sodium pentobarbital (120-125 mg/kg) at 28 days following the instillation of BL or saline.

Morphologic Evaluation: four rats from each group were used for morphological evaluation. After the trachea was cannulated, a thoracotomy was performed and the pulmonary vasculature was ligated at the base of the heart. Lungs were fixed by airway instillation with a cacodylate-buffered glutaraldehyde-paraformaldehyde fixative (400 mOsm, pH 7.0) at 30 cm of H₂O pressure lungs were fixed for at least 2 h, and blocks of tissue were cut from at least two sagittal slabs from the left, right cranial and right caudal lobes of each lung using the fractionator method to obtain a stratified sampling of lung tissue. Each slab was dehydrated in 95% ethanol and embedded in paraffin. Two sections from each lobe were cut and stained with hematoxylin and eosin. A lesion was defined as alveolitis consisting of thickened intra-alveolar septa resulting from edematous swelling, fibrosis or the presence of inflammatory cells in either interstitium or airways.

Processing of Lungs for Biochemical Assay: lungs of each animal designated for biochemical studies were perfused in situ through the right side of the heart with 20 mL ice-cold saline, and then quickly frozen in liquid nitrogen before storing at −80° C. Subsequently, the frozen lungs were thawed and homogenized in 0.1 M KCl, 0.02 M Tris HCl buffer (pH 7.6) with a Polytron homogenizer. After thoroughly mixing the homogenate, its total volume (10-12 mL) was recorded. Samples were divided into aliquots and stored at −80° C.

Statistical Analysis of Data: the biochemical data are expressed on the basis of per lung. Expression of the data on a per lung basis avoids the artificial lowering of the values in BL-treated animals due to presence of proteins of extra-pulmonary origin. All values are reported as the mean ± standard error (SE). The data were compared within the three groups using two-way analysis of variance (SIGMASTAT) and Student-Newman-Keuls method. A value of p≦0.05 was considered the level of biological significance.

Results:

1) Total protein content, mononuclear cell counting and LDH activities from rat lung tissues in SA-, BL- and baicalein-treated groups

Total protein content, mononuclear cell counting and LDH activities from rat lung tissues in SA-, BL- and baicalein-treated groups are shown in table 5.

TABLE 5
Assays of Bronchoalveolar Lavage Fluid
from BL + Baicalein, BL and Vehicle treated groups

| | | Mononuclear | | |
|---|---|---|---|---|
| | | cells | | |
| Groups | n | (×10⁴/ml) | LDH (U/L) | Protein(g/L) |
| Vehicle | 10 | 69.0 ± 26.8 | 1310.54 ± 299.12 | 0.51 ± 0.20 |
| BL | 10 | 745.1 ± 344.7 | 2356.52 ± 611.22** | 2.55 ± 0.65** |
| BL + | 10 | 501.0 ± 310.2 | 1389.61 ± 526.34^{#} | 1.95 ± 0.35** |
| Baicalein | | | | |

**p < 0.01 vs. Vehicle:
^{#}p < 0.05 vs. BL
Note:
LDH denotes Lactate Dehydrogenase.
BL denotes Bleomycin.

2) Histopathological examination for alveolitis and pulmonary fibrosis from lung tissues in SA-, BL- and baicalein-treated groups Histopathological examination for alveolitis and pulmonary fibrosis from lung tissues in SA-, BL- and baicalein-treated groups are shown in table 6.

3) Fibrotic score: A dramatic effect of oral baicalein was seen through an improvement in the remarkable difference in fibrotic score when data from single animals are tabled in table 6.

TABLE 6
Histopathological Examination in BL + Baicalein,
BL and Vehicle treated groups

| | | Alveolitis | | | | Pulmonary | | | |
|---|---|---|---|---|---|---|---|---|---|
| | | Score | | | | Fibrosis Score | | | |
| Groups | n | 0 | I | II | III | 0 | I | II | III |
| Vehicle | 10 | 10 | 0 | 0 | 0 | 10 | 0 | 0 | 0 |
| BL | 10 | 0 | 0 | 7 | 23 | 0 | 0 | 8 | 2 |
| BL + Baicalein | 10 | 0 | 8 | 2 | 0 | 0 | 7 | 3 | 0 |

X² test: There are statistically significant reduction (p < 0.01) in the amount of alveolitis and pulmonary fibrosis in rats receiving baicalein as compared to placebo (BL) control group.
Note:
BL denotes Bleomycin.

4) Toxicity: LD50 for baicalein is 14 g/kg.
   Discussion:

Despite many recent advances in the development of new forms of therapy, idiopathic pulmonary fibrosis still remains a highly lethal disease with a prognosis similar to that of lung cancer. Most of the drugs currently employed to treat this disease have debilitating systemic toxicity [Giri, Lung Biol in Health and Disease, 1995; 80:777-886]. Although in vitro studies at the cellular and biochemical levels are essential in elucidating the mechanism of fibrosis, in vivo studies using reproducible animal models of lung fibrosis are still needed to develop and evaluate the efficacy of new compounds for their potential antifibrotic effects. We have consistently demonstrated that treatment with baicalein offers a marked protection against lung fibrosis in a single dose BL-rat model. Baicalein is orally effective since its antifibrotic effect is obtained by gavage, with very low toxicity (LD50=11 g/kg). The present study was carried out to find out whether or not baicalein would retard the progression of BL-induced lung fibrosis once it has already started. In order to answer this question, we employed a three-dose BL-rat model of lung fibrosis. In this model, baicalein was fed by gavage starting after the instillation of the BL and continued throughout the study. It is interesting that this regimen of baicalein treatment suppressed the BL-induced increases in the connective tissue and inflammation reactivity of the lungs in rats in the BT group as revealed biochemically and cellularly by reduced levels of lung total protein and reduced activities of lung LDH and mononuclear cell counting as compared to rats treated with BL in the BL group. In addition, the histopathological findings also support the biochemical findings in the sense that lungs from rats in the BT group had considerably less extracellular fibers and mononuclear cell alveolitis than the rats in the BL group that had multifocal lesions containing an accumulation of extracellular fibers and a cellular infiltrate of predominantly mononuclear cells.

Regardless of the mechanisms, the results of the present study indicate that oral intake of baicalein following the initiation of lung injury had beneficial effects by ameliorating lung fibrosis. This conclusion is based on the findings that baicalein treatment minimized the BL-induced lung inflammation and fibrosis in the BT group as evaluated by biochemical measurements of decreased total protein and mononuclear cell counting levels and decreased activities of LDH (see table 5) and by histopathological findings of reduced lung lesions (see table 6). In view of these findings, it is tempting to speculate that baicalein another newly discovered compound has a great therapeutic potential for not only preventing, but also treating lung fibrosis in the initial stages of its development.

Each value represents the mean ±SEM of 10 animals. The abbreviations stand for: SA=saline installed; BL=Bleomycin-treated; BT=BL-instilled with Baicalein-treated. Student's T value was 2.43, with P less than 0.02 (highly significant statistically).

### 12. EXMPLE: THE EFFICACY OF BAICALEIN ON HEPATIC CIRRHOSIS IN CCL4-INDUCED RATS

Hepatic fibrosis is characterized by an increased accumulation of extracellular matrix proteins (ECM), mainly collagens. This excessive collagen deposition has been attributed mainly to excess in the synthesis (fibrogenesis), though evidence also suggests a role for down-regulation of collagenolytic mechanisms [Friedman. N Engl J Med 1993; 328(25):1828-1835.]. In Mexico, similarly as in many countries, heavy consumption of alcohol strongly correlates with occurrence of cirrhosis [Mather. Sleisenger MH, Fordtrans JS, editors. 6^{th} ed. Gastrointestinal and liver diseases, vol. 2. Philadelphia, PA: Saunders, 1998. pp. 1199-1214.]. However, in recent years viral infections, mainly by Hepatitis C virus, are becoming major etiologic agents. Most of the treatments have been oriented at suppressing or inactivating the harmful agent. Nonetheless, in many clinical settings this cannot be achieved and the disease progresses to cirrhosis and its complications. In order to find out the usefulness of a putative remedy for cirrhosis, adequate experimental models are desirable to run pre-clinical studies. Liver cirrhosis induced by chronic CCl₄ administration to rats, represents adequate experimental model of cirrhosis amenable to test curative therapies [Recknagel. Trends Pharmacol Sci 1983;4:129-131]. A continuously growing list of drugs have been tested as antifibrogenic agents [Friedman. Hepatology 2000;32(6): 1403-1438.]. Among them are glucocorticoids, colchicine, silymarin, sho-koto-shu, and interferon-α [Friedman. Hepatology 2000;32(6):1403-1438, Bueno J Hepatol 2000;33(6):915-925.], which are effective to some degree in animals as well as in humans. Nevertheless, conclusive evidence concerning efficacy has proven elusive. Recently, sophisticated biotechnological approaches, i.e. Gene Therapy protocols [Snlgado, Mol Ther 2000;2(6):545-551; Ueki, Nat Med 1999;5(2):226-230; Rudolph, Science 2000;287: 1253-1258.] have been instrumented and have shown exciting data on efficacy on fibrosis reversion and liver cell regeneration, although critical aspects of safety on human use have been raised.

Baicalin, a newly developed anti-fibrotic agent has proven effective in vivo in this invention for preventing and resolving the accumulation of fibrous tissue in experimental models of lung fibrosis. Besides the efficacy on pulmonary, the compound was also proved to have the effect on CCl₄ model of experimental liver cirrhosis. Material and methods

Animals and method of baicalin dosing: Fibrosis was induced by chronic CCl₄ administration to rats. Sprague-Dawley rats weighing 200-240 g received three doses a week i.p. of a mixture 1:6 of CCl₄-mineral oil for the first week; then the second week the ratio was 1:5, the third week 1:4, and the 4^{th}-8^{th} weeks the ratio was 1:3 [Armendariz Biochim Biophys Acta 1997;1353:241-252, Armendariz Hepatology 1991;14:895-900.]. CCl₄ intoxication was stopped and cirrhotic animals were then fed by gavage either with the baicalin (n=10) at the dosage of 450 mg/kg or saline (n=10) as a control for 3 more weeks.

Statistical analysis: Results relative to the number of experiments indicated, are expressed as mean ±SD. Statistical analyses were perfomed using Student's t-test.

Preparation of liver homogenates: Rats were killed at indicated times and liver homogenates were prepared from 150 mg of tissue as described [Gao Hum Gene Ther 1999;10:911-922.] and kept at −70° C. At the same time, serum samples were obtained and kept at −20° C. until used. Total protein levels in serum were determined using Bradford assay of protein quantification [Bradford Anal Biochem 1976;72:248-254.].

Biochemical assays: Blood was drawn from control and experimental cirrhotic animals at the moment of sacrifice and serum transaminases alanine aminotransferase (ALT), aspartate aminotransferase (AST) and alkaline phosphatase (ALP) were determined in an automated Sincron-7 machine.

Hydroxyproline content biochemical determinations: Liver samples were obtained at the moment of sacrifice and 150 mg were subjected to acid hydrolysis to determine the amount of hydroxyproline according to Rojkind and coworkers [Rojkind Anal Biochem 1974; 57:1-7].

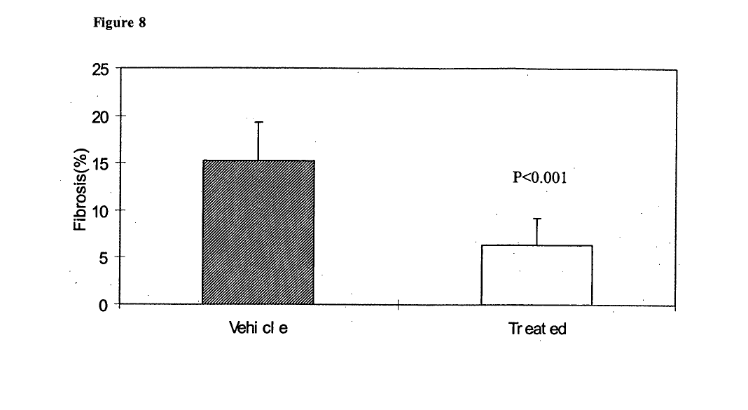

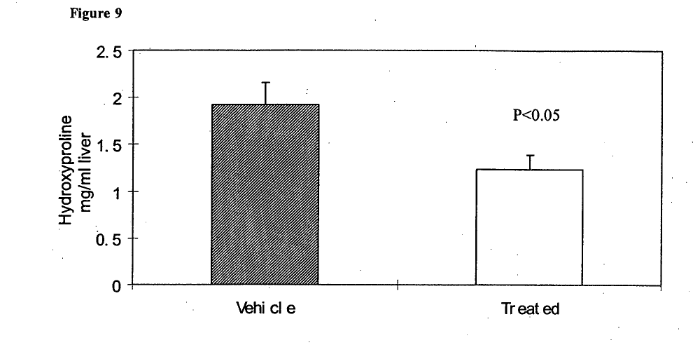

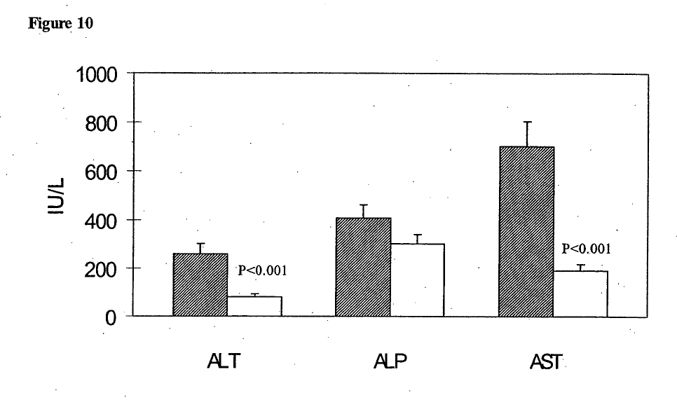

Results and discussion: In the experimental model, rats were injured for 8 weeks with CCl₄, at which time hepatotoxin was discontinued, and then treated for 3 weeks with either daily administrations of 450 mg of baicalin per kilogram, or saline. A dramatic effect of oral baicalin was seen through an improvement in the remarkable difference in fibrotic index when data from single animals are tabled (gray bars vs. black bars in FIG. 8). The gavaged rats displayed a statistically significant 40% liver fibrosis reduction as compared with their control counterparts which were give saline only (n=10) Biochemical determinations of hydroxyproline demonstrated to be significantly lower in baicalin-treated rat livers (FIG. 9). FIG. 10 illustrates functional hepatic tests for each rat in the study, which showed a strong improvement. Notably, AST decreased over 3-fold in Pirfenidone-treated animals (P<0.001). ALT decreased over 2-fold (P<0.001).

All references in the specification to publications such as to issued patents, published patent applications, publications in scientific journals, textbooks and treatises, are hereby incorporated by reference in their entirety for all purposes.

1. A method for the reparation of or prophylaxis against fibrotic lesional tissue, the method comprising:
administering to a mammal a pharmaceutical composition comprising one or more compounds of formula I 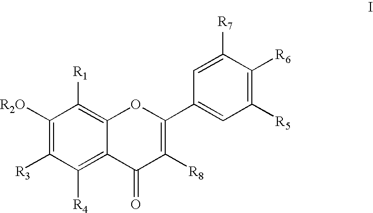
formula I
or a prodrug thereof or a pharmaceutically acceptable salts thereof, in admixture with a pharmaceutically acceptable carrier
wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen, hydroxy, methyl or a straight or branched C₁-C₅ alkoxy;
R₆ is hydrogen, hydroxy, methyl or a straight or branched C₁-C₅ alkoxy;
R₇ is hydrogen, hydroxy, methyl or a straight or branched C₁-C₅ alkoxy;
R₈ is hydrogen, hydroxy or a straight or branched C₁-C₅ alkoxy; and
R₂ is hydrogen, a straight or branched C₁-C₅ alkyl, a protected or unprotected monosaccharide (pyranose or furanose), disaccharide, trisaccharide and their analogues or derivates (sugar alcohol, sugar acid) such as glucose, glucuronic acid, galactose, mannose, allose, idose, allulose, altrose, gulose, etagatose, talose, ribose, arabinose, xylose, lyxose, sorbose, amylomaltose, cellulose, lactose, sucrose.

2. The method of claim 1, wherein the compound is selected from the group consisting of
(2S,3S,4S,5R,6S)-6-(5,6-dihydroxy-4-oxo-2-phenyl-4H-chromen-7-yloxy)-tetrahydro-3,4,5-t rihydroxy-2H-pyran-2-carboxylic acid,
5,6-dihydroxy-7-methoxy-2-phenyl-4H-chromen-4-one,
(2S,3S,4S,5R,6S)-6-(5,6-dihydroxy-2-(4-hydroxyphenyl)-4-oxo-4H-chromen-7-yloxy)-tetrahydroydro-3,4,5-trihydroxy-2H-pyran-2-carboxylic acid,
5,6,7-trihydroxy-2-phenyl-4H-chromen-4-one,
5,6,7-trihydroxy-2-(4-hydroxyphenyl)-4H-chromen-4-one,
5,6,7-trihydroxy-2-(3,4-dihydroxyphenyl)-4H-chromen-4-one,
5,6-dihydroxy-7-methoxy-2-(4-methoxyphenyl)-4H-chromen-4-one,
5,6,7-trihydroxy-2-(4-methoxyphenyl)-4H-chromen-4-one,
5,6-dihydroxy-2-(3,4-dihydroxyphenyl)-7-methoxy-4H-chromen-4-one,
5,6-dihydroxy-2-(4-hydroxy-3-methoxyphenyl)-7-methoxy-4H-chromen-4-one,
5,6-dihydroxy-2-(3-hydroxy-4-methoxyphenyl)-7-methoxy-4H-chromen-4-one,
7-((2S,3R,4R,5R,6S)-tetrahydro-3,4,5-trihydroxy-6-methyl-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
5,6-dihydroxy-2-(4-hydroxyphenyl)-7-methoxy-4H-chromen-4-one,
5,6,7-trihydroxy-2-(4-hydroxy-3-methoxyphenyl)-4H-chromen-4-one,
7-((2S,3S,4R,5S)-tetrahydro-3,4-dihydroxy-5-((R)-1-hydroxyethyl)furan-2-yloxy)-5,6-dihydroxy-2-phenyl-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-hydroxyphenyl)-4H-chromen-4-one,
(2S,3S,4S,5R)-6-(5,6-dihydroxy-4-oxo-2-phenyl-4H-chromen-7-yloxy)-tetrahydro-3,4-trihydroxy-2H-pyran-2-carboxylic acid,
(2R,3R,4R,5S,6R)-6-(5,6-dihydroxy-2-(4-hydroxyphenyl)-4-oxo-4H-chromen-7-yloxy-tetrahydro-3,4,5-trihydroxy-2H-pyran-2-carboxylic acid,
7-((2S,3S,4R,5R,6S)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
(2R,3R,4R,5S,6R)-6-(5,6-dihydroxy-2-(3,4-dihydroxyphenyl)-4-oxo-4H-chromen-7-yloxy)-tetrahydro-3,4,5-trihydroxy-2H-pyran-2-carboxylic acid,
(2R,3R,4R,5S,6R)-6-(5,6-dihydroxy-2-(4-hydroxyphenyl)-4-oxo-4H-chromen-7-yloxy)-tetrahydro-3,4,5-trihydroxy-2H-pyran-2-carboxylic acid,
7-((2S,3S,4S,5S,6R)-tetrahydro-3,4,5-trihydroxy-6-methyl-2H-pyran-2-yloxy)-5,6-dihydroxy-2-(4-methoxyphenyl)-4H-chromen-4-one,
(2S,3S,4S,5R,6R)-5,6-dihydroxy-2-(4-hydroxyphenyl)-4-oxo-4H-chromen-7-yl tetrahydro-3,4,5,6-tetrahydroxy-2H-pyran-2-carboxylate,
7-((2R,3S,4S,5R,6R)-tetrahydro-3,4,5,6-tetrahydroxy-2H-pyran-2-yloxy)-5,6-dihydrox-2-(4-hydroxyphenyl)-4H-chromen-4-one,
7-((2S,3R,4S,5R)-tetrahydro-3,4,5-trihydroxy-5-(hydroxymethyl)furan-2-yloxy)-5,6-dihydroxy-2-(4-dihydroxyphenyl)-4H-chromen-4-one,
7-((2S,3R,4R,5R)-tetrahydro-3,4-dihydroxy-5-((R)-1,2-dihydroxyethyl)furan-2-yloxy)-5,6-dihydroxy-2-phenyl-4H-chromen-4-one,
5,6-dihydroxy-7-methoxy-2-(3,4-dimethoxyphenyl)-4H-chromen-4-one,
(2S,3S,4S,5R,6S)-methyl-6-(5,6-dihydroxy-4-oxo-2-phenyl-4H-chromen-7-yloxy)-tetrahydro-3,4,5-trihydroxy-2H-pyran-2-carboxylate,
7-((2S,3R,4S,5S,6R)-tetrahydro-3,4,5-trihydroxy-6-(hydroxymethyl)-2H-pyran-2-yloxy)-5,6-dihydroxy-2-phenyl-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-hydroxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
7-((2S,3R,4R,5R,6S)-tetrahydro-3,4,5-trihydroxy-6-methyl-2H-pyran-2-yloxy)-5,6-dihydroxy-2-(4-hydroxyphenyl)-4H-chromen-4-one,
(2S,3S,4S,5R)-5,6-dihydroxy-2-(3,4-dihydroxyphenyl)-4-oxo-4H-chromen-7-yl tetrahydro-3,4,5,6-tetrahydroxy-2H-pyran-2-carboxylate,
(2S,3S,4S,5R)-5,6-dihydroxy-2-(4-hydroxy-3-methoxyphenyl)-4-oxo-4H-chromen-7-yl tetrahydro-3,4,5,6-tetrahydroxy-2H-pyran-2-carboxylate,
7-((2S,3R,4R)-tetrahydro-3,4-dihydroxy-4-(hydroxymethyl)furan-2-yloxy)-5,6-dihydroxy-2-(3,4-dihydroxyphenyl)-4H-chromen-4-one,
(2R,3R,4S,R,6S)-6-(5,6-dihydroxy-4-oxo-2-phenyl-4H-chromen-7-yloxy)-tetrahydro-3,4,5-trihydroxy-2H-pyran-2-carboxylic acid,
(2S,3S,4S,5R,6S)-6-(5,6-dihydroxy-2-(3,4-dihydroxyphenyl)-4-oxo-4H-chromen-7-yloxy)-tetrahydro-3,4,5-trihydroxy-2H-pyran-2-carboxylic acid,
3,5,7-trihydroxy-2-(3,4-dihydroxyphenyl)-4H-chromen-4-one,
5,7-dihydroxy-2-phenyl-4H-chromen-4-one,
5,7-dihydroxy-6-methyl-2-phenyl-4H-chromen-4-one,
5,7-dihydroxy-8-methoxy-2-phenyl-4H-chromen-4-one,
5,7-dihydroxy-2-(4-hydroxyphenyl)-4H-chromen-4-one,
5,7-dihydroxy-2-(3,4-dihydroxyphenyl)-4H-chromen-4-one,
3,5,7-trihydroxy-2-(4-hydroxyphenyl)-4H-chromen-4-one,
6,7-dihydroxy-2-phenyl-4H-chromen-4-one,
6,7-dihydroxy-2-(4-hydroxyphenyl)-4H-chromen-4-one,
6,7-dihydroxy-2-(2,3-dihydroxyphenyl)-4H-chromen-4-one,
6,7-dihydroxy-2-(2,4-dihydroxyphenyl)-4H-chromen-4-one,
6,7-dihydroxy-2-(2,5-dihydroxyphenyl)-4H-chromen-4-one,
6,7-dihydroxy-2-(2,6-dihydroxyphenyl)-4H-chromen-4-one,
6,7-dihydroxy-2-(3,5-dihydroxyphenyl)-4H-chromen-4-one,
6,7-dihydroxy-2-(3,4-dihydroxyphenyl)-4H-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-5,6-dihydroxy-2-(4-hydroxy-phenyl)-chromen-4-one,
7-[3,4-dihydroxy-6-methyl-5-(3,4,5-trihydroxy-tetrahydro-pyran-2-yloxy)-tetrahydro-pyran-2-yloxy]-5,6-dihydroxy-2-(4-hydroxyphenyl)-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-5,6-dihydroxy-2-(4-methoxy-phenyl)-chromen-4-one,
5,6-dihydroxy-2-(4-hydroxy-phenyl)-7-[3,4,5-trihydroxy-6-(3,4,5-trihydroxy-6-methyl-tetrah ydro-pyran-2-yloxymethyl)-tetrahydropyran-2-yloxy]-chromen-4-one,
7-[4,5-dihydroxy-6-hydroxymethyl-3-(3,4,5-trihydroxy-tetrahydropyran-2-yloxy)-tetrahydro-pyran-2-yloxy]-2-(3,4-dihydroxy-phenyl)-5,6-dihydroxy-chromen-4-one,
2-(3,4-dihydroxy-phenyl)-5,6-dihydroxy-7-[3,4,5-trihydroxy-6-(3,4,5-trihydroxy-6-methyl-tetrahydro-pyran-2-yloxymethyl)-tetrahydropyran-2-yloxy]-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3,4-dihydroxy-phenyl)-5,6-dihydroxy-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3,4-dihydroxy-phenyl)-5,6-dihydroxy-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3-methoxy-4-hydroxy -phenyl)-5,6-dihydroxy-chromen-4-one,
2-(3,4-dihydroxy-phenyl)-5,6-dihydroxy-7-[3,4,5-trihydroxy-6-(3,4,5-trihydroxy-6-hydroxy methyl-tetrahydro-pyran-2-yloxymethyl)-tetrahydro-pyran-2-yloxy]-chromen-4-one, and
2-(3,4-dihydroxy-phenyl)-7-[4,5-dihydroxy-3-(3,4,5-trihydroxytetrahydro-pyran-2-yloxy)-tetrahydro-pyran-2-yloxy]-5,6-dihydroxychromen-4-one or combinations thereof.

3. The method of claim 1, wherein R₁ is hydrogen, R₂ is hydrogen, R₃ is hydrogen, R₄ is hydroxy, R₅ is hydroxy, R₆ is hydroxy, R₇ is hydrogen, and R₈ is hydroxy to give a compound of formula IV.

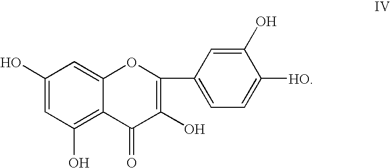

4. The method of claim 1, wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen, hydroxy, methyl or a straight or branched C₁-C₅ alkoxy;
R₆ is hydrogen, hydroxy, methyl or a straight or branched C₁-C₅ alkoxy;
R₇ is hydrogen, hydroxy, methyl or a straight or branched C₁-C₅ alkoxy;
R₈ is hydrogen; and
R₂ is hydrogen, a straight or branched C₁-C₅ alkyl, a protected or unprotected monosaccharide, disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

5. The method of claim 4, wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen;
R₆ is hydrogen;
R₇ is hydrogen;
R₈ is hydrogen; and
R₂ is hydrogen, a straight or branched C₁-C₅ alkyl, a protected or unprotected monosaccharide, disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

6. The method of claim 5, wherein
R₁ is hydrogen;
R₃ is hydrogen, hydroxy or a straight or branched C₁-C₅ alkoxy;
R₄ is hydrogen, hydroxy or a straight or branched C₁-C₅ alkoxy;
R₅ is hydrogen;
R₆ is hydrogen;
R₇ is hydrogen;
R₈ is hydrogen; and
R₂ is hydrogen, a straight or branched C₁-C₅ alkyl, a protected or unprotected monosaccharide, disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

7. The method of claim 6, wherein
R₁ is hydrogen;
R₃ is hydrogen, hydroxy;
R₄ is hydrogen, hydroxy;
R₅ is hydrogen;
R₆ is hydrogen;
R₇ is hydrogen;
R₈ is hydrogen;
R₂ is hydrogen, a straight or branched C₁-C₅ alkyl, a protected or unprotected monosaccharide disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

8. The method of claim 7, wherein
R₁ is hydrogen.
R₃ is hydroxy.
R₄ is hydroxy.
R₅ is hydrogen.
R₆ is hydrogen.
R₇ is hydrogen.
R₈ is hydrogen.
R₂ is hydrogen, a straight or branched C₁-C₅ alkyl, a protected or unprotected monosaccharide, a disaccharide, a trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

9. The method of claim 8, wherein
R₁ is hydrogen;
R₃ is hydroxy;
R₄ is hydroxy;
R₅ is hydrogen;
R₆ is hydrogen;
R₇ is hydrogen;
R₈ is hydrogen; and
R₂ is hydrogen to give baicalein represented by structure CM101 below 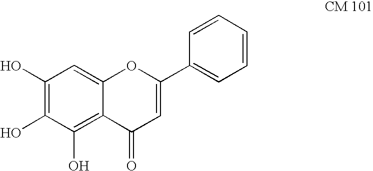
5,6,7-trihydroxy-2-phenyl-4H-chromen-4-one.

10. The method of claim 8, wherein
R₁ is hydrogen;
R₃ is hydroxy;
R₄ is hydroxy;
R₅ is hydrogen;
R₆ is hydrogen;
R₇ is hydrogen;
R₈ is hydrogen; and
R₂ is β-D-glucopyranosiduronate to give Baicalin represented by structure CM105 below 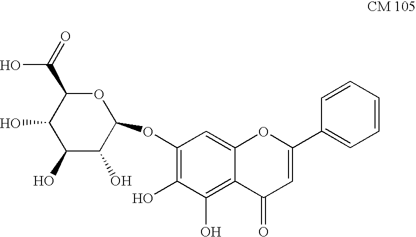
(2S,3S,4S,5R,6S)-6-(5,6-dihydroxy-4-oxo-2-phenyl-4H-chromen-7-yloxy)-trihydroxy-2H-pyran-2-carboxylic acid.

11. A pharmaceutical composition comprising one or more compounds of formula I 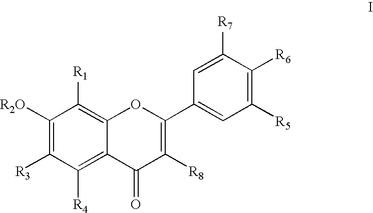
formula I
or a prodrug thereof or a pharmaceutically acceptable salt, hydrate, or solvate thereof, in admixture with a pharmaceutically acceptable carrier
wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen, hydroxy, methyl or a straight or branched C₁-C₅ alkoxy;
R₆ is hydrogen, hydroxy, methyl or a straight or branched C₁-C₅ alkoxy;
R₇ is hydrogen, hydroxy, methyl or a straight or branched C₁-C₅ alkoxy;
R₈ is hydrogen, hydroxy or a straight or branched C₁-C₅ alkoxy; and
R₂ is hydrogen, a straight or branched C₁-C₅ alkyl, a protected or unprotected monosaccharide (pyranose or furanose), disaccharide, trisaccharide and their analogues or derivates (sugar alcohol, sugar acid) such as glucose, glucuronic acid, galactose, mannose, allose, idose, allulose, altrose, gulose, tagatose, talose, ribose, arabinose, xylose, lyxose, sorbose, amylomaltose, cellulose, lactose, sucrose.

12. The pharmaceutical composition of claim 11, wherein the compound is selected from the group consisting of
(2S,3S,4S,5R,6S)-6-(5,6-dihydroxy-4-oxo-2-phenyl-4H-chromen-7-yloxy)-tetrahydro-3,4,5-trihydroxy-2H-pyran-2-carboxylic acid,
5,6-Dihydroxy-7-methoxy-2-phenyl-4H-chromen-4-one,
(2S,3S,4S,5R,6S)-6-(5,6-dihydroxy-2-(4-hydroxyphenyl)-4-oxo-4H-chromen-7-yloxy)-tetrahydro-3,4,5-trihydroxy-2H-pyran-2-carboxylic acid,
5,6,7-Trihydroxy-2-phenyl-4H-chromen-4-one,
5,6,7-Trihydroxy-2-(4-hydroxyphenyl)-4H-chromen-4-one,
5,6,7-Trihydroxy-2-(3,4-dihydroxyphenyl)-4H-chromen-4-one,
5,6-Dihydroxy-7-methoxy-2-(4-methoxyphenyl)-4H-chromen-4-one,
5,6,7-Trihydroxy-2-(4-methoxyphenyl)-4H-chromen-4-one,
5,6-Dihydroxy-2-(3,4-dihydroxyphenyl)-7-methoxy-4H-chromen-4-one,
5,6-Dihydroxy-2-(4-hydroxy-3-methoxyphenyl)-7-methoxy-4H-chromen-4-one,
5,6-Dihydroxy-2-(3-hydroxy-4-methoxyphenyl)-7-methoxy-4H-chromen-4-one,
7-((2S,3R,4R,5R,6S)-tetrahydro-3,4,5-trihydroxy-6-methyl-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
5,6-Dihydroxy-2-(4-hydroxyphenyl)-7-methoxy-4H-chromen-4-one,
5,6,7-Trihydroxy-2-(4-hydroxy-3-methoxyphenyl)-4H-chromen-4-one,
7-((2S,3S,4R,5S)-tetrahydro-3,4-dihydroxy-5-((R)-1-hydroxyethyl)furan-2-yloxy)-5,6-dihydroxy-2-phenyl-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-hydroxyphenyl)-4H-chromen-4-one,
(2S,3S,4S,5R)-6-(5,6-dihydroxy4-oxo-2-phenyl-4H-chromen-7-yloxy)-tetrahydro-3,4,5-trihydroxy-2H-pyran-2-carboxylic acid,
(2R,3R,4R,5S,6R)-6-(5,6-dihydroxy-2-(4-hydroxyphenyl)-4-oxo-4H-chromen-7-yloxy)-tetrahydro-3,4,5-trihydroxy-2H-pyran-2-carboxylic acid,
7-((2S,3S,4R,5R,6S)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
(2R,3R,4R,5S,6R)-6-(5,6-dihydroxy-2-(3,4-dihydroxyphenyl)-4-oxo-4H-chromen-7-yloxy)-tetrahydro-3,4,5-trihydroxy-2H-pyran-2-carboxylic acid,
(2R,3R,4R,5S,6R)-6-(5,6-dihydroxy-2-(4-hydroxyphenyl)-4-oxo-4H-chromen-7-yloxy)-tetrahydro-3,4,5-trihydroxy-2H-pyran-2-carboxylic acid,
7-((2S,3S,4S,5S,6R)-tetrahydro-3,4,5-trihydroxy-6-methyl-2H-pyran-2-yloxy)-5,6-dihydroxy-2-(4-methoxyphenyl)-4H-chromen-4-one,
(2S,3S,4S,5R,6R)-5,6-dihydroxy-2-(4-hydroxyphenyl)-4-oxo-4H-chromen-7-yl tetrahydro-3,4,5,6-tetrahydroxy-2H-pyran-2-carboxylate,
7-((2R,3S,4S,5R,6R)-tetrahydro-3,4,5,6-tetrahydroxy-2H-pyran-2-yloxy)-5,6-dihydroxy-2-(4-hydroxyphenyl)-4H-chromen-4-one,
7-((2S,3R,4S,5R)-tetrahydro-3,4,5-trihydroxy-5-(hydroxymethyl)furan-2-yloxy)-5,6-drihydroxy-2-(3,4-dihydroxyphenyl)-4H-chromen-4-one,
7-((2S,3R,4R,5R)-tetrahydro-3,4-dihydroxy-5-((R)-1,2-dihydroxyethyl)furan-2-yloxy)-5,6-dihydroxy-2-phenyl-4H-chromen-4-one, 5,6-dihydroxy-7-methoxy-2-(3,4-dimethoxyphenyl)-4H-chromen-4-one,
(2S,3S,4S,5R,6S)-methyl-6-(5,6-dihydroxy-4-oxo-2-phenyl-4H-chromen-7-yloxy)-tetrahydro-3,4,5-trihydroxy-2H-pyran-2-carboxylate,
7-((2S,3R,4S,5S,6R)-tetrahydro-3,4,5-trihydroxy-6-(hydroxymethyl)-2H-pyran-2-yloxy)-5,6-dihydroxy-2-phenyl-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-hydroxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
7-((2S,3R,4R,5R,6S)-tetrahydro-3,4,5-trihydroxy-6-methyl-2H-pyran-2-yloxy)-5,6-dihydroxy-2-(4-hydroxyphenyl)-4H-chromen-4-one,
(2S,3S,4S,5R)-5,6-dihydroxy-2-(3,4-dihydroxyphenyl)-4-oxo4H-chromen-7-yl tetrahydro-3,4,5,6-tetrahydroxy-2H-pyran-2-carboxylate,
(2S,3S,4S,5R)-5,6-dihydroxy-2-(4-hydroxy-3-methoxyphenyl)-4-oxo4H-chromen-7-yl tetrahydro-3,4,5,6-tetrahydroxy-2H-pyran-2-carboxylate,
7-((2S,3R,4R)-tetrahydro-3,4-dihydroxy4-(hydroxymethyl)furan-2-yloxy)-5,6-dihydroxy-2-(3,4-dihydroxyphenyl)-4H-chromen-4-one,
(2R,3R,4S,5R,6S)-6-(5,6-dihydroxy4-oxo-2-phenyl-4H-chromen-7-yloxy)-tetrahydro-3,4,5-trihydroxy-2H-pyran-2-carboxylic acid,
(2S,3S,4S,5R,6S)-6-(5,6-dihydroxy-2-(3,4-dihydroxyphenyl)-4-oxo-4H-chromen-7-yloxy)-tetrahydro-3,4,5-trihydroxy-2H-pyran-2-carboxylic acid,
3,5,7-trihydroxy-2-(3,4-dihydroxyphenyl)-4H-chromen-4-one,
5,7-dihydroxy-2-phenyl-4H-chromen-4-one,
5,7-dihydroxy-6-methyl-2-phenyl-4H-chromen-4-one,
5,7-dihydroxy-8-methoxy-2-phenyl-4H-chromen-4-one,
5,7-dihydroxy-2-(4-hydroxyphenyl)-4H-chromen-4-one,
5,7-dihydroxy-2-(3,4-dihydroxyphenyl)-4H-chromen-4-one,
3,5,7-trihydroxy-2-(4-hydroxyphenyl)-4H-chromen-4-one,
6,7-dihydroxy-2-phenyl-4H-chromen-4-one,
6,7-dihydroxy-2-(4-hydroxyphenyl)-4H-chromen-4-one,
6,7-dihydroxy-2-(2,3-dihydroxyphenyl)-4H-chromen-4-one,
6,7-dihydroxy-2-(2,4-dihydroxyphenyl)-4H-chromen-4-one,
6,7-dihydroxy-2-(2,5-dihydroxyphenyl)-4H-chromen-4-one,
6,7-dihydroxy-2-(2,6-dihydroxyphenyl)-4H-chromen-4-one,
6,7-dihydroxy-2-(3,5-dihydroxyphenyl)-4H-chromen-4-one,
6,7-dihydroxy-2-(3,4-dihydroxyphenyl)-4H-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-5,6-dihydroxy-2-(4-hydroxy-phenyl)-chromen-4-one,
7-[3,4-dihydroxy-6-methyl-5-(3,4,5-trihydroxy-tetrahydro-pyran-2-yloxy)-tetrahydro-pyran-2-yloxy]-5,6-dihydroxy-2-(4-hydroxyphenyl)-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-5,6-dihydroxy-2-(4-methoxy-phenyl)-chromen-4-one,
5,6-dihydroxy-2-(4-hydroxy-phenyl)-7-[3,4,5-trihydroxy-6-(3,4,5-trihydroxy-6-methyl-tetrahydro-pyran-2-yloxymethyl)-tetrahydropyran-2-yloxy]-chromen-4-one,
7-[4,5-dihydroxy-6-hydroxymethyl-3-(3,4,5-trihydroxy-tetrahydropyran-2-yloxy)-tetrahydro-pyran-2-yloxy]-2-(3,4-dihydroxy-phenyl)-5,6-dihydroxy-chromen-4-one,
2-(3,4-dihydroxy-phenyl)-5,6-dihydroxy-7-[3,4,5-trihydroxy-6-(3,4,5-trihydroxy-6-methyl-tetrahydro-pyran-2-yloxymethyl)-tetrahydropyran-2-yloxy]-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3,4-dihydroxy-phenyl)-5,6-dihydroxy-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3,4-dihydroxy-phenyl)-5,6-dihydroxy-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3-methoxy-4-hydroxy -phenyl)-5,6-dihydroxy-chromen-4-one,
2-(3,4-dihydroxy-phenyl)-5,6-dihydroxy-7-[3,4,5-trihydroxy-6-(3,4,5-trihydroxy-6-hydroxy methyl-tetrahydro-pyran-2-yloxymethyl)-tetrahydro-pyran-2-yloxy]-chromen-4-one, and
2-(3,4-dihydroxy-phenyl)-7-[4,5-dihydroxy-3-(3,4,5-trihydroxytetrahydro-pyran-2-yloxy)-tetrahydro-pyran-2-yloxy]-5,6-dihydroxychromen-4-one or combinations thereof.

13. The pharmaceutical composition of claim 11, wherein R₁ is hydrogen, R₂ is hydrogen, R₃ is hydrogen, R₄ is hydroxy, R₅ is hydroxy, R₆ is hydroxy, R₇ is hydrogen, and R₈ is hydroxy to give a compound of formula IV.

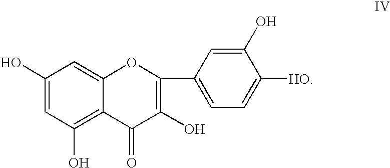

14. The pharmaceutical composition of claim 13, wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen, hydroxy, methyl or a straight or branched C₁-C₅ alkoxy;
R₆ is hydrogen, hydroxy, methyl or a straight or branched C₁-C₅ alkoxy;
R₇ is hydrogen, hydroxy, methyl or a straight or branched C₁-C₅ alkoxy;
R₈ is hydrogen; and
R₂ is hydrogen, a straight or branched C₁-C₅ alkyl, a protected or unprotected monosaccharide, disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

15. The pharmaceutical composition of claim 14, wherein
R₁ is hydrogen, hydroxy or a straight or branched C₁-C₅ alkoxy;
R₃ is hydrogen, hydroxy or a straight or branched C₁-C₅ alkoxy;
R₄ is hydrogen, hydroxy or a straight or branched C₁-C₅ alkoxy;
R₅ is hydrogen;
R₆ is hydrogen;
R₇ is hydrogen;
R₈ is hydrogen; and
R₂ is hydrogen, a straight or branched C₁-C₅ alkyl, a protected or unprotected monosaccharide, disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

16. The pharmaceutical composition of claim 15, wherein
R₁ is hydrogen;
R₃ is hydrogen, hydroxy or a straight or branched C₁-C₅ alkoxy;
R₄ is hydrogen, hydroxy or a straight or branched C₁-C₅ alkoxy;
R₅ is hydrogen;
R₆ is hydrogen;
R₇ is hydrogen;
R₈ is hydrogen; and
R₂ is hydrogen, a straight or branched C₁-C₅ alkyl, a protected or unprotected monosaccharide, disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

17. The pharmaceutical composition of claim 16, wherein
R₁ is hydrogen;
R₃ is hydrogen, hydroxy;
R₄ is hydrogen, hydroxy;
R₅ is hydrogen;
R₆ is hydrogen;
R₇ is hydrogen;
R₈ is hydrogen;
R₂ is hydrogen, a straight or branched C₁-C₅ alkyl, a protected or unprotected monosaccharide disaccharide, trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

18. The pharmaceutical composition of claim 17, wherein
R₁ is hydrogen.
R₃ is hydroxy.
R₄ is hydroxy.
R₅ is hydrogen.
R₆ is hydrogen.
R₇ is hydrogen.
R₈ is hydrogen.
R₂ is hydrogen, a straight or branched C₁-C₅ alkyl, a protected or unprotected monosaccharide, a disaccharide, a trisaccharide and their analogues or derivates selected from the group consisting of glucose, glucuronic acid, galactose, mannose, allose, idose, allulose, altrose, gulose, tagatose, talose, ribose, arabinose, xylose, lyxose, sorbose, amylomaltose, cellulose, lactose, and sucrose.

19. The pharmaceutical composition of claim 18, wherein
R₁ is hydrogen;
R₃ is hydroxy;
R₄ is hydroxy;
R₅ is hydrogen;
R₆ is hydrogen;
R₇ is hydrogen;
R₈ is hydrogen; and
R₂ is hydrogen to give baicalein represented by structure CM101 below 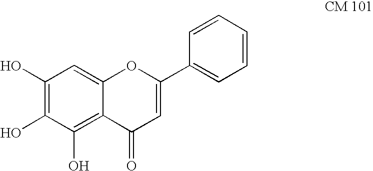
5,6,7-trihydroxy-2-phenyl-4H-chromen-4-one.

20. The pharmaceutical composition of claim 18, wherein
R₁ is hydrogen;
R₃ is hydroxy;
R₄ is hydroxy;
R₅ is hydrogen;
R₆ is hydrogen;
R₇ is hydrogen;
R₈ is hydrogen; and
R₂ is β-D-glucopyranosiduronate to give baicalin represented by structure CM105 below 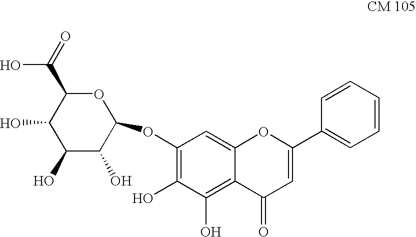
(2S,3S,4S,5R,6S)-6-(5,6-dihydroxy-4-oxo-2-phenyl-4H-chromen-7-yloxy)-tetrahydro-3,4,5-trihydroxy-2H-pyran-2-carboxylic acid.

21. A method for the reparation of or prophylaxis against fibrotic lesional tissue the method comprising administering to a mammal extracts or fractions of extracts from botanicals selected from the group consisting of Scutellaria baicalensis Georgi, Scutellaria scordifolia Fisch, Oroxylum indicum(L.) Vent, and Plantago major L. pharmaceutical, wherein the extracts comprise a compound selected from the group consisting of
(2S,3S,4S,5R,6S)-6-(5,6-dihydroxy-4-oxo-2-phenyl-4H-chromen-7-yloxy)-tetrahydro-3,4,5-trihydroxy-2H-pyran-2-carboxylic acid,
5,6-dihydroxy-7-methoxy-2-phenyl-4H-chromen-4-one,
(2S,3S,4S,5R,6S)-6-(5,6-dihydroxy-2-(4-hydroxyphenyl)-4-oxo-4H-chrommen-7-yloxy)-tetrahydro-3,4,5-trihydroxy-2H-pyran-2-carboxylic acid,
5,6,7-trihydroxy-2-phenyl-4H-chromen-4-one,
5,6,7-trihydroxy-2-(4-hydroxyphenyl)-4H-chromen-4-one,
5,6,7-trihydroxy-2-(3,4-dihydroxyphenyl)-4H-chromen-4-one,
5,6-dihydroxy-7-methoxy-2-(4-methoxyphenyl)-4H-chromen-4-one,
5,6,7-trihydroxy-2-(4-methoxyphenyl)-4H-chromen-4-one,
5,6-dihydroxy-2-(3,4-dihydroxyphenyl)-7-methoxy-4H-chromen-4-one,
5,6-dihydroxy-2-(4-hydroxy-3-methoxyphenyl)-7-methoxy-4H-chromen-4-one,
5,6-dihydroxy-2-(3-hydroxy-4-methoxyphenyl)-7-methoxy-4H-chromen-4-one,
7-((2S,3R,4R,5R,6S)-tetrahydro-3,4,5-trihydroxy-6-methyl-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-methoxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
5,6-dihydroxy-2-(4-hydroxyphenyl)-7-methoxy-4H-chromen-4-one,
5,6,7-trihydroxy-2-(4-hydroxy-3-methoxyphenyl)-4H-chromen-4-one,
7-((2S,3S,4R,5S)-tetrahydro-3,4-dihydroxy-5-((R)-1-hydroxyethyl)furan-2-yloxy)-5,6-dihydroxy-2-phenyl-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-hydroxyphenyl)-4H-chromen-4-one,
(2S,3S,4S,5R)-6-(5,6-dihydroxy4-oxo-2-phenyl-4H-chromen-7-yloxy)-tetrahydro-3,4,5trihydroxy-2H-pyran-2-carboxylic acid,
(2R,3R,4R,5S,6R)-6-(5,6-dihydroxy-2-(4-hydroxyphenyl)-4-oxo4H-chromen-7-yloxy)-tetrahydro-3,4,5-trihydroxy-2H-pyran-2-carboxylic acid,
7-((2S,3S,4R,5R,6S)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
(2R,3R,4R,5S,6R)-6-(5,6-dihydroxy-2-(3,4-dihydroxyphenyl)-4-oxo-4H-chromen-7-yloxy)-tetrahydro-3,4,5-trihydroxy-2H-pyran-2-carboxylic acid,
(2R,3R,4R,5S,6R)-6-(5,6-dihydroxy-2-(4-hydroxyphenyl)-4-oxo-4H-chromen-7-yloxy)-tetrahydro-3,4,5-trihydroxy-2H-pyran-2-carboxylic acid,
7-((2S,3S,4S,5S,6R)-tetrahydro-3,4,5-trihydroxy-6-methyl-2H-pyran-2-yloxy)-5,6-dihydroxy-2-(4-methoxyphenyl)-4H-chromen-4-one,
(2S,3S,4S,5R,6R)-5,6-dihydroxy-2-(4-hydroxyphenyl)-4-oxo-4H-chromen-7-yl tetrahydro-3,4,5,6-tetrahydroxy-2H-pyran-2-carboxylate,
7-((2R,3S,4S,5R,6R)-tetrahydro-3,4,5,6-tetrahydroxy-2H-pyran-2-yloxy)-5,6-dihydroxy-2-(4-hydroxyphenyl)-4H-chromen-4-one,
7-((2S,3R,4S,5R)-tetrahydro-3,4,5-trihydroxy-5-(hydroxymethyl)furan-2-yloxy)-5,6-dihydroxy-2-(3,4-dihydroxyphenyl)-4H-chromen-4-one,
7-((2S,3R,4R,5R)-tetrahydro-3,4-dihydroxy-5-((R)-1,2-dihydroxyethyl)furan-2-yloxy)-5,6-dihydroxy-2-phenyl-4H-chromen-4-one,
5,6-dihydroxy-7-methoxy-2-(3,4-dimethoxyphenyl)-4H-chromen-4-one,
(2S,3S,4S,5R,6S)-methyl-6-(5,6-dihydroxy-4-oxo-2-phenyl-4H-chromen-7-yloxy)-tetrahydro-3,4,5-trihydroxy-2H-pyran-2-carboxylate,
7-((2S,3R,4S,5S,6R)-tetrahydro-3,4,5-trihydroxy-6-(hydroxymethyl)-2H-pyran-2-yloxy)-5,6-dihydroxy-2-phenyl-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-hydroxyphenyl)-4H-chromen-4-one,
7-((2S,3R,4S,5S,6R)-tetrahydro-3,4,5-trihydroxy-6-(hydroxymethyl)-2H-pyran-2-yloxy)-5,6-dihydroxy-2-(4-methoxyphenyl)-4H-chromen-4-one, 7-((2S,3R,4S,5S,6R)-tetrahydro-3,4,5-trihydroxy-6-(hydroxymethyl)-2H-pyran-2-yloxy)-5,6-dihydroxy-2-(3,4-dihydroxyphenyl)-4H-chromen-4-one,
7-((2S,3R,4R,5R,6S)-tetrahydro-3,4,5-trihydroxy-6-methyl-2H-pyran-2-yloxy)-5,6-dihydroxy-2-(4-hydroxyphenyl)-4H-chromen-4-one,
(2S,3S,4S,5R)-5,6-dihydroxy-2-(3,4-dihydroxyphenyl)-4-oxo4H-chromen-7-yl tetrahydro-3,4,5,6-tetrahydroxy-2H-pyran-2-carboxylate,
(2S,3S,4S,5R)-5,6-dihydroxy-2-(4-hydroxy-3-methoxyphenyl)-4-oxo4H-chromen-7-yl tetrahydro-3,4,5,6-tetrahydroxy-2H-pyran-2-carboxylate,
7-((2S,3R,4R)-tetrahydro-3,4-dihydroxy4-(hydroxymethyl)furan-2-yloxy)-5,6-dihydroxy-2-(3,4-dihydroxyphenyl)-4H-chromen-4-one,
(2R,3R,4S,5R,6S)-6-(5,6-dihydroxy-4-oxo-2-phenyl-4H-chromen-7-yloxy)-tetrahydro-3,4,5-trihydroxy-2H-pyran-2-carboxylic acid,
(2S,3S,4S,5R,6S)-6-(5,6-dihydroxy-2-(3,4-dihydroxyphenyl)-4-oxo4H-chromen-7-yloxy)-tetrahydro-3,4,5-trihydroxy-2H-pyran-2-carboxylic acid,
3,5,7-trihydroxy-2-(3,4-dihydroxyphenyl)-4H-chromen-4-one,
5,7-dihydroxy-2-phenyl-4H-chromen-4-one,
5,7-dihydroxy-6-methyl-2-phenyl-4H-chromen-4-one,
5,7-dihydroxy-8-methoxy-2-phenyl-4H-chromen-4-one,
5,7-dihydroxy-2-(4-hydroxyphenyl)-4H-chromen-4-one,
5,7-dihydroxy-2-(3,4-dihydroxyphenyl)-4H-chromen-4-one,
3,5,7-trihydroxy-2-(4-hydroxyphenyl)-4H-chromen-4-one,
6,7-dihydroxy-2-phenyl-4H-chromen-4-one,
6,7-dihydroxy-2-(4-hydroxyphenyl)-4H-chromen-4-one,
6,7-dihydroxy-2-(2,3-dihydroxyphenyl)-4H-chromen-4-one,
6,7-dihydroxy-2-(2,4-dihydroxyphenyl)-4H-chromen-4-one,
6,7-dihydroxy-2-(2,5-dihydroxyphenyl)-4H-chromen-4-one,
6,7-dihydroxy-2-(2,6-dihydroxyphenyl)-4H-chromen-4-one,
6,7-dihydroxy-2-(3,5-dihydroxyphenyl)-4H-chromen-4-one,
6,7-dihydroxy-2-(3,4-dihydroxyphenyl)-4H-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-5,6-dihydroxy-2-(4-hydroxy-phenyl)-chromen-4-one,
7-[3,4-dihydroxy-6-methyl-5-(3,4,5-trihydroxy-tetrahydro-pyran-2-yloxy)-tetrahydro-pyran-2-yloxy]-5,6-dihydroxy-2-(4-hydroxyphenyl)-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-5,6-dihydroxy-2-(4-methoxy-phenyl)-chromen-4-one, 5,6-dihydroxy-2-(4-hydroxy-phenyl)-7-[3,4,5-trihydroxy-6-(3,4,5-trihydroxy-6-methyl-tetrahydro-pyran-2-yloxymethyl)-tetrahydropyran-2-yloxy]-chromen-4-one,
7-[4,5-dihydroxy-6-hydroxymethyl-3-(3,4,5-trihydroxy-tetrahydropyran-2-yloxy)-tetrahydro-pyran-2yloxy]-2-(3,4-dihydroxy-phenyl)-5,6-dihydroxy-chromen-4-one,
2-(3,4-dihydroxy-phenyl)-5,6-dihydroxy-7-[3,4,5-trihydroxy-6-(3,4,5-trihydroxy-6-methyl-tetrahydro-pyran-2-yloxymethyl)-tetrahydropyran-2-yloxy]-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3,4-dihydroxy-phenyl)-5,6-dihydroxy-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3,4-dihydroxy-phenyl)-5,6-dihydroxy-chromen-4-one,
7-[4,5-dihydroxy-6-hydroxymethyl-3-(3,4,5-trihydroxy-6-hydroxymethyl-tetrahydro-pyran-2-yloxy)-tetrahydro-pyran-2-yloxy]-2-(3-methoxy-4-hydroxy -phenyl)-5,6-dihydroxy-chromen-4-one,
2-(3,4-dihydroxy-phenyl)-5,6-dihydroxy-7-[3,4,5-trihydroxy-6-(3,4,5-trihydroxy-6-hydroxy methyl-tetrahydro-pyran-2-yloxymethyl)-tetrahydro-pyran-2-yloxy]-chromen-4-one, and
2-(3,4-dihydroxy-phenyl)-7-[4,5-dihydroxy-3-(3,4,5-trihydroxytetrahydro-pyran-2-yloxy)-tetrahydro-pyran-2-yloxy]-5,6-dihydroxychromen-4-one or combinations thereof.

22. The method of claim 1, wherein said fibrotic lesional tissue is associated with a condition selected from the group consisting of pulmonary fibrosis, benign prostate hypertrophy, coronary infarcts, cerebral infarcts, myocardiac fibrosis, musculoskeletal fibrosis, post-surgical adhesions, liver cirrhosis, renal fibrotic disease, fibrotic vascular disease, scleroderma, Alzheimer's disease, diabetic retinopathy, and skin lesions.

23. The method of claim 1, wherein said pharmaceutical composition is administered as a capsule, a tablet, a powder, a granule, a syrup, an injectable fluid, a cream, an ointment, an inhalable fluid, an eye drop, a suppository, or a pill.

24. The method of claim 1, wherein the route of administration is intravenous.

25. The method of claim 1, wherein the route of administration is intranasal.

26. The method of claim 1, wherein the mammal is a human.

28. The method of claim 1, wherein the mammal is a household pet or a livestock animals selected from the group consisting of cow, horse, sheep, pig, cat, dog, mouse, rat, rabbit, chicken, turkey, duck, goose, and quail.

29. The method of claim 1, wherein the pharmaceutical composition comprises one or more compounds in an amount of from about 1% to 90% w/w.

30. The method of claim 1, wherein the pharmaceutical composition comprises one or more compounds in an amount of about 0.01 milligrams to about 900 milligrams per kilogram of the body weight.

31. The method of claim 30, wherein the pharmaceutical composition comprises one or more compounds in an amount of about 0.01 picogram to 1 milligrams per kilogram of the body weight.
